(19) United States
(12) Patent Application Publication
(10) Pub. No.: US 2003/0032674 A1
(43) Pub. Date: Feb. 13, 2003

(54) Use of unsaturated fatty acids to treat severe inflammatory diseases

(76) Inventor: Daniel H. Hwang, Baton Rouge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/218,226

(22) Filed: Aug. 13, 2002

Related U.S. Application Data
(60) Provisional application No. 60/312,007, filed on Aug. 13, 2001.

(51) Int. Cl. A61K 31/202

(52) U.S. Cl. 514/560

(57) ABSTRACT

The infusion of unsaturated fatty acids in a solution essentially free of saturated fatty acids can effectively ameliorate the symptoms of sepsis, septic shock and other severe inflammatory diseases caused by the presence of compounds from bacteria, yeast and mycobacteria. Saturated fatty acids through activation of the Toll-receptors TLR4 and TLR2 induce the expression of proinflammatory cytokines, iNOS, and COX-2, important compounds in the cascading response leading to septic shock. In contrast, unsaturated fatty acids, including omega-3, omega-6, and omega-9 fatty acids, inhibit the expression of COX-2 stimulated by saturated fatty acids, with docosahexaenoic acid being the most effective inhibitor. These surprising results indicate that an effective treatment of sepsis or other severe inflammatory diseases is the infusion of polyunsaturated fatty acids that are essentially free of saturated fatty acids. The unsaturated fatty acids can be infused either (1) bound to serum albumin in a solution of a single fatty acid or in a solution of a mixture of unsaturated fatty acids; or (2) combined with glycerol to produce a triglyceride.

The development of this invention was partially funded by the Government under grants from the National Institute of Health (R01 DK-41868 and CA-75613), from American Institute for Cancer Research (98A0978), and from the United States Department of Agriculture (97-37200-4258). The Government has certain rights in this invention.

This invention pertains to the administration of unsaturated fatty acids that are essentially free of saturated fatty acids for the treatment of severe inflammatory disorders, for example, sepsis, septic shock, or septicemia.

Septic shock and multiple-organ failure are catastrophic consequences of an invasive infection. Septic shock has been estimated to occur in more than 500,000 cases per year in the United States alone. Septic shock is the most common cause of death in non-coronary, intensive care units. As more antibiotic-resistant strains of bacteria evolve, the incidence of septic shock is expected to increase. Overall mortality rates from septic shock range from 30% to 90%. Aggressive antibiotic treatment and timely surgical intervention are the main therapies, but in many cases are insufficient. The search for new drug therapies has not been successful. R. Stone, “Search for Sepsis Drugs Goes On Despite Past Failures,” Science, vol. 264, pp. 365-367 (1994). See, e.g., A. Fein, “Treatment of Severe Systemic Inflammatory Response Syndrome and Sepsis with a Novel Bradykinin Antagonist, Deltibant (CP-0127),” J. Am. Med. Assoc., vol. 277, pp. 482-487 (1997), reporting small, but not statistically significant, improvements in 28-day mortality compared to placebo when the compound deltibant was administered to human patients suffering systemic inflammatory response syndrome and presumed sepsis. (Deltibant is a dimer of two peptides joined to one another by a linker.)

Sepsis is a morbid condition induced by a toxin, the introduction or accumulation of which is most commonly caused by infection or trauma. The initial symptoms of sepsis typically include chills, profuse sweating, irregularly remittent fever, prostration, and the like; followed by persistent fever, hypotension leading to shock, neutropenia, leukopenia, disseminated intravascular coagulation, acute respiratory distress syndrome, and multiple organ failure.

Lipopolysaccharide (LPS) is believed to be the principal agent of Gram-negative bacteria responsible for inducing sepsis syndrome, which includes septic shock, systemic inflammatory response syndrome, and multiorgan failure. LPS, also known as endotoxin, is atoxic component of the outer membrane of Gram-negative microorganisms (e.g., Escherichia coli, Klebsiella pneumonia, Pseudomonas aeruginosa). Compelling evidence supports the toxic role of LPS; all pathophysiological effects noted in humans during Gram-negative sepsis can be duplicated in laboratory animals by injection of purified LPS. The mechanism by which LPS activates responsive cells is complex and not fully understood. The host response to Gram-negative bacterial infection depends on effector cell recognition of the bacteria, LPS, or both, and involves both serum proteins and cell membrane receptors. When bacteria and LPS are removed via endocytosis and phagocytosis by reticuloendothial cells, concomitant activation of the host immune response by LPS results in the secretion of cytokines by activated macrophages, which in turn can trigger the exaggerated host responses associated with septic shock.

Other microbial products of bacteria, fungi, and mycobacteria can trigger the inflammatory response that can lead to sepsis or septic shock. See K. A. Heldwein et al., “Recent Advances in the biology of Toll-like receptors,” Mod. Asp. Immunobiol., vol. 1, pp. 249-252 (2001).

The normal immune response begins when neutrophils squeeze through the blood-vessel walls searching for bacterial pathogens in the surrounding tissue. Neutrophils can kill bacteria directly by releasing toxic chemicals or enzymes, such as elastase or collagenase. The neutrophils also attract other leukocytes to the area, including lymphocytes, macrophages, and monocytes, the last two of which release powerful immune-response activators called cytokines. The cytokines, in turn, stimulate more immune cell activity and increase the number of cells coming to the area by making neighboring blood-vessel walls more permeable. Then, as the number of bacteria decreases, other cytokines signal to bring the normal immune response to an end.

If the cutoff mechanism fails, however, sepsis can begin. In sepsis, humoral and cellular mediators cascade in a process that becomes at least temporarily independent of the underlying infection. Excess neutrophils and macrophages are drawn to the site of infection, releasing excess immune-stimulating cytokines, eventually triggering the release of substances that damage the blood-vessel wall. More monocytes and macrophages come to the site and release more cytokines. Eventually, the blood vessels are so damaged and leaky that blood pressure falls and the blood can no longer supply nutrients to the body's organs. Entire organs can begin to shut down. Many patients die after losing the function of two or more organs.

Two cytokines that play an important role in sepsis are interleukin-1 (IL-1) and tumor necrosis factor-alpha (TNFα). These two polypeptides can raise body temperature, increase the expression of adhesion molecules on neutrophils and endothelial cells (promoting adhesion of leukocytes), stimulate the production of vasodilating prostaglandins (thus increasing the permeability of blood vessels), trigger the release of other cytokines, stimulate neutrophils, and activate fibroblasts. All these processes enhance the probability of organ failure seen in severe septicemia. Drug therapies that target only one of these two cytokines have proved ineffective. See Stone (1994). Drug therapies that are effective against general inflammatory responses have not proven to be effective against the cascading acute inflammation that produces septicemia.

Other important cytokines, chemokines, and proteins having proinflammatory activity include interferon-gamma (IFN-γ), interleukin-6 (IL-6), macrophage chemotactic protein (MCP), inducible nitric oxide synthetase (iNO S), mitogen- activated protein kinases (MAPKs), macrophage inflammatory protein (MIP), cytokine-inducible neutrophil chemoattractant (KC/CINC), tissue factor (TF), granulocyte-macrophage-colony stimulating factor (GM-CSF) and phosphotyrosine phosphatase (PTPase).

Prostaglandins are also involved in the proinflammatory response; e.g., prostaglandins increase the permeability of the blood-vessel wall. Cyclooxygenase (COX; prostaglandin endoperoxide synthase) catalyzes the conversion of arachidonic acid to prostaglandin (PG) endoperoxide (PGH2), which is the rate-limiting step in prostaglandin biosynthesis. Two isoforms of COX have been cloned from animal cells: the constitutively expressed COX-1, and the mitogen-inducible COX-2. Prostaglandins produced as a result of the activation of COX-1 may have physiological functions such as the antithrombogenic action of prostacyclin released by the vascular endothelium, and the cytoprotective effect of PGs produced by the gastric mucosa. COX-2 is expressed following the activation of cells by various proinflammatory agents including cytokines, endotoxin, and other mitogens. These observations suggest that COX-2 instead of COX-1 may be responsible for inducing production of the prostaglandins involved in inflammation. Only a few pharmacological agents have been identified that suppress the expression of COX-2 without affecting COX-1, for example, radicicol, sesquiterpene lactone, rofecoxib (also known as “refecoxib”); etoricoxib (also known as “MK-663”); NS-398; DuP-697; SC-58125; DFU; L745,337; RS 57067; celecoxib (also known as “SC-58635”); valdecoxib; meloxicam; flosulide; nimesulide; and parecoxib. See U.S. Pat. Nos. 6,180,651; 6,004,994; 5,972,950; 5,859,036; 5,905,089; 5,731,343; 5,866,596; and 5,686,460; International Application Nos. WO 01/52897 A2; WO 01/28548 A1; and WO 01/15687 A1.

The pathogenesis of Gram-negative septic shock is presumably generated by the excess stimulation of host cells by bacterial LPS endotoxin. Such stimulation leads to the expression and release of pro-inflammatory marker gene products and lipid mediators, which in turn initiate a chain of events leading to systemic toxicity. Initial recognition of LPS by the host cells is required to initiate the cascading response. See S. H. Rhee and D. Hwang, “Murine TOLL-like receptor 4 confers lipopolysaccharide responsiveness as determined by activation of NFκB and expression of the inducible cyclooxygenase,” Journal of Biological Chemistry, vol. 275, pp. 34035-34040 (2000).

Toll-like receptors (TLRs) play a critical role in the detection of microbial infection and the induction of inflammatory and immune responses against pathogen associated molecular patterns. The activation of TLRs leads to the induction of NFκB activation, the expression of inflammatory cytokines, TNFα, and the expression of COX-2. See A. Aderem et al., “Toll-like receptors in the induction of the innate immune response,” Nature, vol. 406, pp.782-787 (2000). Ten members of the TLR family have so far been identified in human and mouse and these TLRs are ubiquitously expressed in human tissues. However, ligands for these TLRs have not been fully identified. Genetic and biochemical evidence have demonstrated that TLR4 confers the responsiveness to lipopolysaccharide (LPS) derived from gram-negative bacteria, the plant product Taxol, heat shock protein-60, and the respiratory syncytial virus coat protein F. TLR2 agonists are bacterial lipoproteins, glycolipids, peptidoglycan (a Gram-positive pathogen component), and zymosan (a yeast cell-wall particle). See Heldwein et al., 2001; A. Ozinsky et al., “The repertoire for pattern recognition of pathogens by the innate immune system is defined by cooperation between Toll-like receptors,” PNAS, vol. 97, pp. 13766-13771 (2000); A. M. Hajjar et al., “Cutting edge: Functional interactions between Toll-like receptor (TLR)2 and TLR1 or TLR6 in response to phenol soluble modulin,” The Journal of Immunology, vol. 166, pp. 15-19 (2001); and L. Alexopoulou et al., “Hyporesponsiveness to vaccination with Borrelia burgdorferi OspA in humans and in TLR1- and TLR2-deficient mice,” Nature Medicine, vol. 8, pp. 878-883 (2002).

Recently, both human and murine Toll-like receptors, e.g., receptor 2 (TLR2) and receptor 4 (TLR4), have been shown to mediate the cellular signaling pathway that leads to increase in production of proinflammatory cytokines and COX-2. Moreover, recent research has shown that TLRs can form either homodimers or heterodimers with other TLRs to recongnize a diverse array of bacterial components. See Helwein et al., 2001; Ozinsky et al., 2000; Hajjar et al., 2001; and Alexopoulou et al., 2002. For example, a functional interaction has been found between TLR2 and either TLR6 or TLR1. The association of TLR6 and TLR2 is known to recognize peptidoglycan, a Gram-positive pathogen component. See Hajjar et al., 2001; and Alexopoulou et al., 2002.

LPS activation of one or more of the Toll-receptor proteins leads to the activation of NFκB, which is required for the full expression of COX-2 from LPS-stimulated macrophages. COX2 expression is induced by various mitogenic stimuli in different cell types. The cis-acting NFκB element is present in the 5′-flanking regions of COX-2 genes of different species. Pro-inflammatory cytokines such as TNFα and IL-1 also activate NFκB and induce COX-2 expression in many cell types. Transcription factor NFκB regulates a diverse array of genes that are involved in innate immune responses and pathogenesis of Gram-negative septic shock. See Rhee and Hwang, 2000.

The lipid A moiety possesses most of the biological activities of LPS. Lipid A of Escherichia coli is comprised of three principal fatty acid species: 3-hydroxymyristic acid (3-OH14:0; about 65 mass %), lauric acid (C12:0; about 15 mass %), and myristic acid (C14:0; about 18 mass %). These acyl-linked saturated fatty acids are subject to hydrolysis by acyloxyacyl hydrolase, and the deacylated LPS loses its endotoxic properties. See Y. Weinrauch et al., “Deacylation of purified lipopolysaccharides by cellular and extracellular components of a sterile rabbit peritoneal, inflammatory exudate,” Infection and Immunity, vol. 67, pp. 3376-3382 (1999). The deacylated LPS also acts as an antagonist against the native LPS.

Most long-chain fatty acids in mammalian cellular lipids are esterfied. Therefore, the concentrations of unesterified fatty acids are believed to be low. However, fatty acids are rapidly released in response to extracellular stimuli by the action of phospholipase A₂, monoacylglycerol lipase, and diacylglycerol lipase. In plasma the average concentrations of free fatty acids and triglycerides in a postabsorptive state are <0.7 mM and <1.8 mM, respectively. These concentrations may be much higher in an absorptive phase after ingestion of a fatty meal. See D. B. Jump et al., “Regulation of gene expression by dietary fat,” Annu. Rev. Nutr., vol. 19, pp. 63-90 (1999). Therefore, blood cells such as monocytes are constantly exposed to relatively high concentrations of free fatty acids. Fatty acids are known to regulate the expression of many genes involved in lipid metabolism and to modulate the activity of signaling molecules such as phospholipase C and protein kinase C. See Jump et al., 1999; and D. Hwang et al., “receptor-mediated signaling pathways: potential targets of modulation by dietary fatty acids,” Am. J. Clin. Nutr., vol. 70, pp. 545-56 (1999). The mechanism by which fatty acids regulate gene expression is not well understood.

Fatty acids and their oxidative metabolites are known to bind and activate peroxisome proliferator-activated receptors (PPARs), the steroid-thyroid super family of nuclear receptors. Two Zn-finger motifs in the DNA binding domain of PPARs bind PPAR response elements (PPREs) located in the 5′-flanking region of PPAR responsive genes. PPARs bind PPRE as a heterodimer with the retinoid X receptor (RXR). Polyunsaturated fatty acids (PUFAs) and other peroxisome proliferators induce peroxisomal β-oxidation and the expression of certain peroxisomal enzymes. See Hwang et al., 1999.

A common problem in critical illness is hypolipidemia, resulting in extremely low plasma concentrations of cholesterol, low densitylipoprotein, and high density lipoprotein. Various lipid preparations have been infused to treat hypolipidemia. In particular, an infusion of a high-density lipoprotein preparation was shown to decrease the LPS-induced production of tumor necrosis factor-α, probably by binding to lipopolysaccharide. See B. R. Gordon et al., “Low lipid concentrations in critical illness: Implications for preventing and treating endotoxemia,” Crit. Care Med., vol. 24, pp. 584-589 (1996).

Human populations with diets high in fish oil, which contain omega-3-polyunsaturated fatty acids (ω-3-PLFAs), were shown to have a lower incidence of thrombosis, coronary heart disease and myocardial infarction. ω-3-PUFAs (or n-3-PUFAs) are long-chain polyunsaturated fatty acids with a double bond between carbon atoms 3 and 4 proximal to the methyl end of the fatty acid. Other families of unsaturated fatty acids include n-6 (e.g., linoleic acid (C18:2n-6) and arachidonic acid (C20:4n-6)), n-7 (e.g., palmitoleic acid (C16:1n-7)), and n-9 (e.g., oleic acid (C18:1n-9)). Naturally occurring species of the n-3 family are eicosapentaenoic acid (EPA), docosahexaenoic acid (DHA), and α-linolenic acid (α-LA). See S. Endres et al., “n-3 Polyunsaturated fatty acids: update 1995,” European Journal of Clinical Investigation, vol. 25, pp. 629-638 (1995). Dietary augmentation with ω-3-PUFAs has reduced the incidence of nosocomial infections and the length of hospital stay for sepsis patients. See A. Heller et al., “Lipid Mediators in Inflammatory Disorders,” Drugs, vol. 55, pp. 487-496 (1998). Dietary supplementation with a fish-oil concentrate resulted in a suppression of interleukin production, tumor necrosis factor, and mononuclear cell proliferation. See S. Endres et al., “The effect of dietary supplementation with n-3 polyunsaturated fatty acids on the synthesis of interleukin-1 and tumor necrosis factor by mononuclear cells,” N. Engl. J. Med., vol. 320, pp. 265-271 (1989); and S. Endres et al., “Dietary supplementation with n-3 fatty acids suppresses interleukin-2 production and mononuclear cell proliferation,” J. Leukoc. Biol., vol. 54, pp. 599-603 (1993).

Because of the undesirable flavor and high cost of fish oil, flaxseed oil was used for dietary supplementation of EPA, along with sunflower oil as a control. However, all of these oils have approximately 10% or more saturated fatty acids. See U.S. Pat. No. 6,008,248. Flaxseed oil contains high concentrations of α-linolenic acid, from which EPA is endogenously formed. Sunflower oil is rich in n-6 polyunsaturated fatty acids. Flaxseed oil, although not as effective as fish oil in increasing plasma concentrations of EPA, was still effective in decreasing the production of tumor necrosis factor (TNFαA) and interleukin-1β(IL-1β). However, a diet rich in sunflower oil did not cause a change in the plasma concentration of EPA or in the production of either TNFα, IL-1β, thromboxane B₂, or prostaglandin E₂. See G. E. Caughey et al., “The effect on human tumor necrosis factor α and interleukin 1β production of diets enriched in n-3 fatty acids from vegetable oil or fish oil,” Am. J. Clin. Nutr., vol. 63, pp. 116-122 (1996).

Incubating mouse macrophages in EPA-rich media indicated that an increase in EPA will decrease macrophage tumor necrosis factor gene transcription by decreasing the activity of NFκB, and will also reduce the production of prostaglandin production by LPS-stimulated macrophages, which cause an increase in COX-2 mRNA transcription through a feedback mechanism. See C.-J. Lo et al., “Fish oil decreases macrophage tumor necrosis factor gene transcription by altering the NFκB activity,” Journal of Surgical Research, vol. 82, pp. 216-221 (1999); and C.-J. Lo et al., “Fish oil augments macrophage cyclooxygenase II (COX-2) gene expression induced by endotoxin,” Journal of Surgical Research, vol. 86, pp. 103-107 (1999).

The effects of other fatty acids on the inhibition of COX-2 have been investigated. See T. Ringbom et al., “COX-2 Inhibitory effects of naturally occurring and modified fatty acids,” J. Nat. Prod., vol. 64, pp. 745-749 (2001). No effect on COX-2 was seen with the saturated fatty acids of stearic acid (SA; 18:0), palmitic acid (PA; 16:0); pentadecanoic acid (PDA; 15:0), and myristic acid (MA; 14:0). In addition, the omega-9 fatty acid (oleic acid (OA; 18:1ω-9) showed no effect. Inhibitory effects on COX-2 were seen with the omega-3 fatty acids of EPA, DHA, and α-LA. Linoleic acid (LA), an omega-6 fatty acid (18:2ω-6), was not as effective in the inhibition of COX-2 as the omega-3 fatty acids.

Infusion of 1,2,3-trieicosapentaenoyl-glycerol (EPA-TG), synthesized from glycerol and free EPA, has been shown to inhibit natural killer cell activity in both mice and humans, to decrease platelet aggregation, and to increase the urinary concentration of both prostaglandin I₃ and thromboxane A₃. See M. Grakaze et al., “Infusion of emulsified trieicosapentaenoyl-glycerol into rabbits -- The effects on platelet aggregation, polymorphonuclear leukocyte adhesion, and fatty acid composition in plasma and platelet phospholipids,” Thrombosis Research, vol. 44, pp. 673-682 (1986); N. Yamashita et al., “Inhibition of natural killer cell activity of human lymphocytes by eicosapentaenoic acid,” Biochem. Biophys. Res. Comm., vol. 138, pp. 1058-1067 (1986); N. Yamashita et al., “Inhibition of natural killer cell activity by eicosapentaenoic acid in vivo and in vitro,” Biochem. Biophys. Res. Comm., vol. 150, pp. 497-505 (1988); and T. Hamazaki et al., “The infusion of trieicosapentaenoyl-glycerol into humans and the in vivo formation of prostaglandin I₃ and thromboxane A₃,” Biochem. Biophys. Res. Comm., vol. 151, pp. 1386-1394 (1988). Additionally, infusion of EPA-TG has been proposed to be useful in prevention of acute organ rejection due to a decrease leukotriene B₄ production. See T. Hamazaki, “Intravenous infusion of n-3 polyunsaturated fatty acids,” P.S.E.M.B., vol. 200, pp. 171-173 (1992).

U.S. Pat. Nos. 5,344,822; 5,506,218; 5,587,366; 5,614,507; and 5,674,855 disclose the treatment of endotoxemia by administration of a mixture of neutral lipids and phospholipids in a composition that is essentially free of peptides and proteins.

U.S. Pat. No. 5,840,757 discloses a dietary lipid emulsion, comprising a mixture of triglycerides containing both saturated and unsaturated long-chain fatty acids.

U.S. Pat. No. 5,993,221 discloses a dietary oil emulsion that comprises about 60% of both omega-3 fatty acid and arachidonic moieties and which is administered to increase immunity in critically ill patients by restoring arachidonic acid levels.

U.S. Pat. No. 5,998,482 discloses a treatment of sepsis or septic shock by administration of synthetic cationic amphiphilic molecules that bind and sequester bacterial LPS. Such synthetic molecules possess one or more lipophilic groups from fatty acids or hydrocarbon substitutents.

U.S. Pat. No. 6,008,248 discloses isotonic lipid emulsions comprising medium chain triglycerides, vegetable oils and fish oil for use as parental nutrition.

None of the above references suggest that administration of saturated fatty acids is detrimental in the treatment of sepsis and should be avoided.

I have discovered that the infusion of unsaturated fatty acids in a solution essentially free of saturated fatty acids can effectively ameliorate the symptoms of sepsis, septic shock and other severe inflammatory diseases caused by the presence of compounds from bacteria, yeast, and mycobacteria. Saturated fatty acids through activation of the Toll-receptors TLR4 and TLR2 induce the expression of proinflammatory cytokines, iNOS, and COX-2, important compounds in the cascading response leading to septic shock. In contrast, unsaturated fatty acids, including omega-3, omega-6, and omega-9 fatty acids, inhibit the expression of COX-2 stimulated by saturated fatty acids, with docosahexaenoic acid being the most effective inhibitor. These surprising results indicate that an effective treatment of sepsis or other severe inflammatory diseases is the infusion of polyunsaturated fatty acids that are essentially free of saturated fatty acids. The unsaturated fatty acids can be infused either (1) bound to serum albumin in a solution of a single fatty acid or in a solution of a mixture of unsaturated fatty acids; or (2) combined with glycerol to produce a triglyceride.

### BRIEF DESCRIPTION OF DRAWINGS

FIG. 1A illustrates a Western blot indicating the effect of a saturated fatty acid lauric acid (C12:0), in concentrations ranging from 1 to 100 μM, on the concentrations of COX-2, iNOS, and IL-1αa expression in a murine macrophage-like cell line (RAW 264.7).

FIG. 1B illustrates the Relative Luciferase Activity (RLA) resulting from a luciferase reporter gene assay for COX-2 used to measure COX-2 activity in a murine macrophage-like cell line (RAW 264.7) treated with various concentrations of the saturated fatty acid lauric acid (C12:0).

FIG. 1C illustrates the Relative Luciferase Activity (RLA) resulting from a luciferase reporter gene assay for COX-2 used to measure COX-2 activity in a murine macrophage-like cell line (RAW 264.7) treated with several saturated fatty acids, especially lauric acid (C12:0) and palmitic acid (C16:0), at a concentration of 75 μM.

FIG. 1D illustrates a Western blot indicating the effect of several unsaturated fatty acids, at a concentration of 75 μM, on the concentration of COX-2 and GAPDH expression in a murine macrophage-like cell line (RAW 264.7).

FIG. 2A illustrates the Relative Luciferase Activity (RLA) resulting from luciferase reporter gene assay for NFκB used to measure NFκB activity in a murine macrophage-like cell line (RAW 264.7) treated with various concentrations of the saturated fatty acid lauric acid (C12:0).

FIG. 2B illustrates the Relative Luciferase Activity (RLA) resulting from luciferase reporter gene assay for COX-2 used to measure COX-2 activity in a murine macrophage-like cell line (RAW 264.7) treated with various combinations of lauric acid (C12:0), a dominant-negative mutant of κBα plasmid, and vector.

FIG. 2C illustrates the Relative Luciferase Activity (RLA) resulting from luciferase reporter gene assay for COX-2 with the COX-2 promoter reporter gene containing either a wild-type NFκB site or a mutated NFκB site, and used to measure COX-2 activity in a murine macrophage-like cell line (RAW 264.7) treated with lauric acid (C12:0).

FIG. 3A illustrates the Relative Luciferase Activity (RLA) resulting from luciferase reporter gene assay for COX-2 with the COX-2 promoter reporter gene that is intact (bases −3201 to +93) or that has a deletion of the PPAR response element (PPRE)-like sequences (bases −1017 to +93), and used to measure COX-2 activity in a murine macrophage-like cell line (RAW 264.7) treated with lauric acid (C12:0).

FIG. 3B illustrates the Relative Luciferase Activity (RLA) resulting from luciferase reporter gene assay for COX-2 with the COX-2 promoter reporter gene that is intact (bases −3201 to +93) or that has a deletion of the PPAR response element (PPRE)-like sequences (bases −1017 to +93), and used to measure COX-2 activity in a murine macrophage-like cell line (RAW 264.7), the cell line co-transfected with a dominant-negative mutant of PPARy plasmid or with just vector, and treated with lauric acid (C12:0).

FIG. 4A illustrates the Relative Luciferase Activity (RLA) resulting from luciferase reporter gene assay for NFκB used-to measure NFκB activity in a murine macrophage-like cell line (RAW 264.7) treated with various combinations of lauric acid (C12:0), a dominant-negative mutant of TLR4 (Toll-like receptor 4), and vector.

FIG. 4B illustrates the Relative Luciferase Activity (RLA) resulting from luciferase reporter gene assay for COX-2 used to measure COX-2 activity in a murine macrophage-like cell line (RAW 264.7) treated with various combinations of lauric acid (C12:0), a dominant-negative mutant of TLR4 (Toll-like receptor 4), and vector.

FIG. 5A illustrates the Relative Luciferase Activity (RLA) resulting from luciferase reporter gene assay for NFκB used to measure NFκB activity in a murine macrophage-like cell line (RAW 264.7) treated with the saturated fatty acid lauric acid (C12:0) and various unsaturated fatty acids.

FIG. 5B illustrates the Relative Luciferase Activity (RLA) resulting from luciferase reporter gene assay for COX-2 used to measure COX-2 activity in a murine macrophage-like cell line (RAW 264.7) treated with the saturated fatty acid lauric acid (C12:0) and various unsaturated fatty acids.

FIG. 6A illustrates the Relative Luciferase Activity (RLA) resulting from luciferase reporter gene assay for COX-2 used to measure COX-2 activity in a murine macrophage-like cell line (RAW 264.7) treated with the unsaturated fatty acid docosahexaenoic acid (DHA; C22:6ω-3) and a constitutively active TLR4 (ΔTLR4).

FIG. 6B illustrates the Relative Luciferase Activity (RLA) resulting from luciferase reporter gene assay for COX-2 used to measure COX-2 activity in a murine macrophage-like cell line (RAW 264.7) treated with the unsaturated fatty acid docosahexaenoic acid (DHA; C22:6ω-3) and a constitutively active MyD88.

FIG. 6C illustrates the Relative Luciferase Activity (RLA) resulting from luciferase reporter gene assay for COX-2 used to measure COX-2 activity in a murine macrophage-like cell line (RAW 264.7) treated with the unsaturated fatty acid docosahexaenoic acid (DHA; C22:6ω-3) and a constitutively active wild-type NFκB-inducing kinase (NIK).

FIG. 7A illustrates a Western blot indicating the effect of an unsaturated fatty acid docosahexaenoic acid (DHA; C22:6ω-3), in concentrations ranging from 5 to 50 μM, on the concentrations of COX-2, iNOS, IL-1a, and GAPDH expression in a murine macrophage-like cell line (RAW 264.7) exposed to LPS.

FIG. 7B illustrates a Western blot indicating the effect of an unsaturated fatty acid docosahexaenoic acid (DHA; C22:6ω-3), in concentrations ranging from 5 to 50 μM, on the concentration of IκBα expression in a murine macrophage-like cell line (RAW 264.7) exposed to LPS.

FIG. 7C illustrates a Western blot indicating the effect of an unsaturated fatty acid docosahexaenoic acid (DHA; C22:6ω-3), in concentrations ranging from 5 to 50 μM, on the concentration of COX-2 and GAPDH expression in a murine macrophage-like cell line (RAW 264.7) treated with TNFα.

FIG. 8A illustrates the Relative Luciferase Activity (RLA) resulting from luciferase reporter gene assay for NFκB used to measure NFκB activity in a murine macrophage-like cell line (RAW 264.7) treated with various concentrations of several unsaturated fatty acids (DHA, docosahexaenoic acid (C22:6n-3); EPA, eicosapentaenoic acid (C20:5n-3); AA, arachidonic acid (C20:4n-6); LA, linoleic acid (C18:2n-6); OA, and oleic acid (C18:1n-9)) and a saturated fatty acid (lauric acid (C12:0)).

FIG. 8B illustrates the Relative Luciferase Activity (RLA) resulting from luciferase reporter gene assay for COX-2 used to measure COX-2 activity in a murine macrophage-like cell line (RAW 264.7) treated with various concentrations of several unsaturated fatty acids (DHA, docosahexaenoic acid (C22:6n-3); EPA, eicosapentaenoic acid (C20:5n-3); AA, arachidonic acid (C20:4n-6); LA, linoleic acid (C18:2n-6); OA, and oleic acid (C18:1n-9)) and a saturated fatty acid (lauric acid (C12:0)).

FIG. 8C illustrates a Western blot indicating the effect of an unsaturated fatty acid docosahexaenoic acid (DHA; C22:6ω-3) and lauric acid (C12:0) on the concentration of COX-2 and Actin expression in a murine macrophage-like cell line (RAW 264.7) exposed to LPS.

FIG. 9A illustrates the Relative Luciferase Activity (RLA) resulting from luciferase reporter gene assay for NFκB with the NFκB promoter reporter gene, and used to measure NFκB activity in a murine macrophage-like cell line (RAW 264.7), the cell line co-transfected with the expression plasmid of consitutively active human TLR4 (CD4-TLR4, and treated with DHA and lauric acid (C12:0).

FIG. 9B illustrates the Relative Luciferase Activity (RLA) resulting from luciferase reporter gene assay for NFκB with the NFκB promoter reporter gene, and used to measure NFκB activity in a murine macrophage-like cell line (RAW 264.7), the cell line co-transfected with either the expression plasmid of consitutively active MyD88 [MyD88(CA)] or wild type NIK [NIK(WT)], and treated with DHA and lauric acid (C12:0).

FIG. 10A illustrates the Relative Luciferase Activity (RLA) resulting from luciferase reporter gene assay for NFκB with the NFκB promoter reporter gene, and used to measure NFκB activity in the 293T cell line, the cell line co-transfected with various expression plasmids as indicated (pcDNA3.1 and pRK were transfected as vector controls for dominant negative (DN) mutants of MyD88 and NIK; WT is wild type), and treated with either vehicle (control) or a synthetic bacterial lipoprotein (PamCAG, 1 μg/ml).

FIG. 10B illustrates the Relative Luciferase Activity (RLA) resulting from luciferase reporter gene assay for NFκB with the NFκB promoter reporter gene, and used to measure NFκB activity in a murine macrophage-like cell line (RAW 264.7), the cell line co-transfected with various expression plasmids as indicated (pcDNA3.1 and pDisplay were transfected as vector controls for dominant negative (DN) mutants of MyD88 and TLR2), and treated with either vehicle (control) or a synthetic bacterial lipoprotein (PamCAG, 1 μg/ml).

FIG. 10C illustrates the Relative Luciferase Activity (RLA) resulting from luciferase reporter gene assay for COX-2 with the COX-2 promoter reporter gene, and used to measure COX-2 activity in a murine macrophage-like cell line (RAW 264.7), the cell line co-transfected with various expression plasmids as indicated (pcDNA3.1 and pDisplay were transfected as vector controls for dominant negative (DN) mutants-of MyD88 and TLR2), and treated with either vehicle (control) or a synthetic bacterial lipoprotein (PamCAG, 1 μg/ml).

FIG. 11A illustrates the Relative Luciferase Activity (RLA) resulting from luciferase reporter gene assay for NFκB used to measure NFκB activity in a murine macrophage-like cell line (RAW 264.7) pretreated for 3 hr with various concentrations of several unsaturated fatty acids (DHA, docosahexaenoic acid (C22:6n-3); EPA, cicosapentaenoic acid (C20:5n-3); AA, arachidonic acid (C20:4n-6); LA, linoleic acid (C18:2n-6); OA, and oleic acid (C18:1n-9)) and a saturated fatty acid (lauric acid (C12:0)), before exposure to a synthetic bacterial lipoprotein (PamCAG, 500 ng/ml).

FIG. 11B illustrates the Relative Luciferase Activity (RLA) resulting from luciferase reporter gene assay for COX-2 used to measure COX-2 activity in a murine macrophage-like cell line (RAW 264.7) pretreated for 3 hr with various concentrations of several unsaturated fatty acids (DHA, docosahexaenoic acid (C22:6n-3); EPA, eicosapentaenoic acid (C20:5n-3); AA, arachidonic acid (C20:4n-6); LA, linoleic acid (C18:2n-6); OA, and oleic acid (C18:1n-9)) and a saturated fatty acid (lauric acid (C12:0)), before exposure to a synthetic bacterial lipoprotein (PamCAG, 500 ng/ml).

FIG. 11C illustrates a Western blot indicating the effect of pretreatment with an unsaturated fatty acid docosahexaenoic acid (DHA; C22:6ω-3) and lauric acid (C12:0) on the concentration of COX-2 and Actin expression in a murine macrophage-like cell line (RAW 264.7) exposed to a synthetic bacterial lipoprotein (PamCAG,).

FIG. 11D illustrates the Relative Luciferase Activity (RLA) resulting from luciferase reporter gene assay for NFκB used to measure NFκB activity in 293T cells co-transfected with TLR2 expression plasmid, and treated with a synthetic bacterial lipoprotein (PamCAG, 100 ng/ml) in the presence or absence of docosahexaenoic acid (C22:6n-3) (DHA) or lauric acid (C12:0).

Sepsis or other severe inflammatory diseases resulting from stimulation of Toll-receptor proteins can be effectively treated with infusions of polyunsaturated fatty acids that are essentially free of saturated fatty acids. This treatment helps stop the physiological mechanisms at the beginning of the sepsis cascade. I have shown that unsaturated fatty acids in concentrations <100 μM are effective in inhibiting the stimulation of Toll- receptor protein, while saturated fatty acids will stimulate the TOLL receptor protein. Because the normal level of free fatty acids in plasma is <700 μM, the amount of unsaturated fatty acid needed is well below the normal level and should not increase the overall plasma free fatty acid concentration to a toxic level. Moreover, during sepsis, total lipid levels in plasma decreases. Without wishing to be bound by this theory, I believe that the total fatty acid concentration in plasma would also decrease in sepsis. Thus, during the septic state, an infusion of an effective amount of unsaturated fatty acids may not necessarily raise the plasma levels of total fatty acids even to the normal level.

The unsaturated fatty acids may, for example, be infused after binding with serum albumin, or they may be combined with glycerol to form a triglyceride. The infusion should be essentially free of saturated fatty acids, either as free fatty acid or as part of a triglyceride. The unsaturated fatty acids can be, for example, omega-3, omega-6, or omega-9 fatty acids, including eicosapentaenoic acid, docosahexaenoic acid, linoleic acid, conjugated linoleic acid, and oleic acid. The most effective unsaturated fatty acid in inhibiting the inflammatory response, as shown below, was docosahexaenoic acid. Plasma unsaturated fatty acids could also be increased by infusion of synthetic phospholipids that contain only unsaturated fatty acids.

The term “essentially free of saturated fatty acids” as used herein implies that the concentration of saturated fatty acids is so minimal, as not to materially affect the basic and novel properties imparted to the unsaturated fatty acids and as not to activate the Toll-receptors and induce the expression of COX-2.

Unsaturated fatty acids may be administered to a patient by any suitable means, e.g., oral or parenteral administration. Parenteral infusions include intramuscular, intravenous, intraarterial, or intraperitoneal administration.

Pharmaceutically acceptable carrier preparations for parenteral administration include sterile, aqueous or non-aqueous solutions, suspensions, and emulsions. Examples of non-aqueous solvents are propylene glycol and polyethylene glycol. Aqueous carriers include water, alcoholic/aqueous solutions, emulsions or suspensions, including saline and buffered media. Parenteral vehicles include sodium chloride solution, Ringer's dextrose, dextrose and sodium chloride, or lactated Ringer's. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

A severe inflammatory disorder treated by the method of the present invention may be associated with expression of COX-2 or proinflammatory agents such as cytokines, TNFα, IL-1, and MAPKS production, for example.

The present invention provides a method of treating or ameliorating a severe inflammatory disorder such as sepsis, endotoxemia, or septic shock, or one or more of the symptoms of sepsis; comprising administering a therapeutically effective amount of unsaturated fatty acids to a subject displaying such symptoms or at risk for developing sepsis. The term “ameliorate” refers to a decrease or lessening of the symptoms of the disorder being treated. The symptoms that may be ameliorated include those associated with a transient increase in the blood level of TNFα, such as fever, hypotension, neutropenia, leukopenia, thrombocytopenia, disseminated intravascular coagulation, adult respiratory distress syndrome, shock, and multiple organ failure. Patients who may benefit from such treatment include those at risk for or those suffering from toxemia, such as endotoxemia resulting from a Gram-negative bacterial infection, venom poisoning, or hepatic failure. In addition, patients having a Gram-positive bacterial, viral, or fungal infection may also display symptoms of sepsis, and may also benefit from the therapeutic method described here.

Patients likely to benefit from the method of the present invention include those suffering from infection by Gram negative bacteria such as E. coli, Haemophilus influenza B, Neisseria meningitides, staphylococci, or pneumococci. Patients at risk for developing sepsis include those suffering from bums, gunshot wounds, renal failure, hepatic failure, trauma, immunodepression (including HIV infection), hematopoietic neoplasias, multiple myeloma, Castleman's disease, or cardiac myxoma.

The term “therapeutically effective amount” as used herein for treatment of sepsis, septicemia, toxic shock, or endotoxemia refers to an amount of a unsaturated fatty acid or a mixture of unsaturated fatty acids, which are essentially free of saturated fatty acids, sufficient to decrease the subject's response to LPS or bacterial lipoprotein, or to decrease the symptoms of sepsis or other severe inflammatory disorder, to a statistically significant degree (p <0.05). The term “therapeutically effective amount” therefore includes, for example, an amount of unsaturated fatty acid sufficient to prevent, and preferably to reduce by at least 50%, and more preferably sufficient to reduce by at least 90%, a clinically significant increase in a patient's plasma level of TNFα. The dosage ranges for the administration of unsaturated fatty acids are those that produce the desired effect. Generally, the dosage will vary with the age, condition, and sex of the patient, and the extent of the infection. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the level of COX-2, NFκB, or TNFα in a patient. A decrease in serum TNFα levels should correlate with recovery of the patient.

In addition, patients at risk for or exhibiting the symptoms of sepsis can be treated by the novel method, substantially simultaneously with the therapeutic administration of other TNFα inhibitors. For example, administering an anti-TNF antibody or a TNFα antagonist can help prevent or ameliorate the symptoms of sepsis. Particularly preferred is the use of an anti-TNF antibody as an active ingredient, such as a monoclonal antibody as described by Tracey et al. Nature, vol. 330, p. 662 (1987).

Moreover, patients could be treated with the novel method substantially simultaneously with the therapeutic administration of-another known compound that is effective against sepsis or that are known COX-2 inhibitors, e.g., radicicol, sesquiterpene lactone, rofecoxib (also known as “refecoxib”); etoricoxib (also known as “MK-663”); NS-398; DuP-697; SC-58125; DFU; L-745,337; RS 57067; celecoxib (also known as “SC-58635”); valdecoxib; meloxicam; flosulide; nimesulide; and parecoxib. See U.S. Pat. Nos. 6,180,651; 6,004,994; 5,972,950; 5,859,036; 5,905,089; 5,731,343; 5,866,596; and 5,686,460; International Application Nos. WO 01/52897 A2; WO 01/28548 A1; and WO 01/15687 A1.

A patient who exhibits the symptoms of sepsis may also be treated with an antibiotic in addition to the treatment with unsaturated fatty acids. Typical antibiotics include an aminoglycoside such as gentamycin, or a beta-lactam such as penicillin or cephalosporin. Therefore, a preferred therapeutic method includes administering a therapeutically effective amount of unsaturated fatty acids substantially simultaneously with administration of a bactericidal amount of an antibiotic. Preferably, administration of unsaturated fatty acids occurs within about 48 hours and preferably within about 2-8 hours, and most preferably, substantially concurrently with administration of the antibiotic.

The term “bactericidal amount” refers to an amount sufficient to achieve a bacteria-killing blood concentration in the patient receiving the treatment. The bactericidal amounts of antibiotics for administration to a human are well known in the art, and as is known in the art, vary with the specific antibiotic and the type of bacterial infection being treated.

The effectiveness of treatment may be monitored by detection methods used in the art, including immunoassays, Northern and Western blot analysis, and RNase protection assays. Examples of immunoassays that may be used to detect and monitor levels of cytokines, chemokines, mitogens, or other proteins affected by unsaturated fatty acids include competitive and non-competitive immunoassays, in either a direct or indirect format, such as a radioimmunoassay (RIA) or a sandwich (immunometric) assay. An immunoassay of a protein may be run in forward mode, reverse mode, or simultaneous modes, including competition immunoassays, and immunohistochemical assays on physiological samples. Monitoring is preferably performed by a forward immunoassay. Those of skill in the art will know, or can readily discern, other immunoassay monitoring formats without undue experimentation.

Solid phase-bound antibody molecules can be bound by adsorption from an aqueous medium, although other modes of fixation, such as covalent coupling or other known means of fixation to a solid matrix maybe used. Preferably, the first antibody molecule is bound to a support before forming an immunocomplex with antigen (e.g., cytokine); however, the immunocomplex may also be formed prior to binding the complex to the solid support.

Non-specific protein binding sites on the surface of the solid phase support are preferably blocked. After adsorption of solid phase-bound antibodies, an aqueous solution of a protein free from interference with the assay—such as bovine, horse, or other serum albumin—that is also free from contamination with the antigen, is admixed with the solid phase to adsorb the admixed protein onto the surface of the antibody-containing solid support at protein binding sites on the surface that are not occupied by the antibody molecule.

A typical aqueous protein solution contains about 2-10 weight percent bovine serum albumin in phosphate-buffered saline (PBS) at a pH about 7-8. The aqueous protein solution-solid support mixture is typically maintained for at least one hour at a temperature of about 37-40° C., and the resulting solid phase is thereafter rinsed free of unbound protein.

The first antibody can be bound to a carrier and used to detect a cytokine or other protein in a sample. Examples of such carriers include glass, polystyrene, polypropylene, polyethylene, dextran, nylon, amyloses, natural and modified celluloses, polyacrylamides, agaroses, and magnetite. The carrier may be soluble or insoluble. Those skilled in the art will know of other suitable carriers for binding antibodies or antigen, or will be able to ascertain such carriers through routine experimentation.

In addition, if desired, an antibody in these immunoassays can be detectably labeled in various ways. There are many different labels and methods of labeling known to those skilled in the art. Examples of the types of labels which can be used in the present invention include enzymes, radioisotopes, fluorescent compounds, colloidal metals, chemiluminescent compounds, and bioluminescent compounds. Those of skill in the art will known of other suitable labels for binding to monoclonal antibodies, or will be able to ascertain such labels through routine experimentation. Furthermore, binding such labels to the antibodies may be performed with routine methods known in the art.

The following examples are intended to illustrate but not limit the invention. While they are typical of those that might be used, other procedures otherwise known to those skilled in the art may alternatively be used.

### EXAMPLE 1

### Materials and Methods

Reagents—All saturated and unsaturated fatty acids were purchased from Nu-Chek (Eslyan, Minn.). Rumenic acid [9(Z), 11(E)-octadecadienoic acid; conjugated linoleic acid (“cLA”)] was purchased from Matreya (Pleasant Gap, Pa.). Lipopolysaccharide (LPS) was purchased from DIFCO (Detroit, Mich.). Bovine serum albumin (BSA, fatty acid free and low endotoxin, Cat.No. A8806) and human recombinant TNFα were purchased from Sigma (St. Louis, Mo.). Polyclonal antibodies for COX-2 were prepared and characterized as described previously. See D. Hwang et al., “Expression of cyclooxygenase-1 and cyclooxygenase-2 in human breast cancer,” J. Natl. Cancer Inst., vol. 90, pp. 455-460 (1998); and S. H. Lee et al., “Selective expression of mitogen-inducible cyclooxygenase in macrophages stimulated with lipopolysaccharide,” J. Biol. Chem., vol. 267, pp. 25934-25938 (1992). A synthetic bacterial lipoprotein (PamCAG: palmitoyl-Cys((RS)-2,3-di(palmitoyloxy)-propyl)-Ala-Gly-OH) was purchased from Bachem (King of Prussia, Pa.). Poly[I:C] was purchased from Amersham Pharmacia Biotech (Piscataway, N.J.). Antibodies for inducible form of nitric-oxide synthase (iNOS) and interleukin-1α(IL-1α) were purchased from Santa Cruz Biotech (Santa Cruz, Calif.). Donkey anti-rabbit immunoglobulin G (IgG) antibodies conjugated to horseradish peroxidase were purchased from Amersham Pharmacia Biotech (Arlington Heights, Ill.). Enhanced chemiluminescence (ECL) Western blotting detection reagents were purchased from Amersham Corp (Piscataway, N.J.). SuperFect Transfection Reagent was purchased from Qiagen (Valencia, Calif.). Luciferase Assay System and P-galactosidase Enzyme System were purchased from Promega (Madison, Wis.). All other reagents were purchased from Sigma unless otherwise described.

Cell culture—RAW 264.7 cells (a murine macrophage-like cell line, ATCC TIB-71), 293T cells (a human embryonic kidney cell line, provided by Sam Lee, Beth Israel Hospital. Boston, Mass.), and HT-29 cells (a human colon adenocarcinoma cell line, ATCC HTB-38) were cultured in Dulbecco's modified Eagle's medium (DMEM) containing 10% (v/v) heat-inactivated fetal bovine serum (FBS, Intergen) and 100 units/ml Penicillin and 100 mg/ml Streptomycin (GIBCO-BRL) at 37° C. in a 5% CO₂/air environment. Cells (2×10⁶) were plated in 60-mm dishes and cultured for an additional 18 hours to allow the number of cells to approximately double. Cells were maintained in serum-poor (0.25% FBS) medium for another 18 hours prior to the treatment with indicated reagents.

Preparation of fatty acids-albumin complexes—All fatty acids were solubilized in ethanol, and then combined with fatty acid-free and low-endotoxin BSA at a molar ratio of 10:1 (fatty acid: albumin) in serum-poor medium (0.25% FBS). Fatty acids-albumin complex solution was freshly prepared prior to each experiment.

Sodium dodecyl sulfate-polyacrylamide gel electrophoresis (SDS-PAGE) and immunoblotting—These procedures were performed as previously described in D. Hwang et al., “Expression of mitogen-inducible cyclooxygenase induced by lipopolysaccharide: mediation through both mitogen-activated protein kinase and NF-kappaB signaling pathways in macrophages,” Biochem. Pharmacol., vol. 54, pp. 87-96 (1997). Briefly, solubilized proteins were subjected to 8% SDS-PAGE for COX-2, iNOS, IL- 1α and GAPDH immunoblot analyses. Following electrophoresis, the gel was transferred to a PVDF membrane and the membrane was blocked to prevent non-specific binding of antibodies in TBS-T [20 mM Tris HCl, 137 mM NaCl, 0.05% (v/v) Tween 20, pH 7.6] containing 5% non-fat dried milk (Carnation). Immunoblotting was performed using respective polyclonal antibodies followed by incubation with anti-rabbit IgG coupled to horseradish peroxidase. The membrane was exposed on an X-ray film (Kodak) using ECL western blot detection reagents (Amersham).

Plasmids and DNA Constructs—All plasmids and constructs were obtained or made as reported in either S. H. Rhee and D. Hwang, “Murine TOLL-like receptor 4 confers lipopolysaccaride responsiveness as determined by activation of NFκB and expression of the inducible cyclooxygenase,” Journal of Biological Chemistry, vol. 275, pp. 34035-34040 (2000); and J. Y. Lee et al., “Saturated fatty acids, but not unsaturated fatty acids, induce the expression of cyclooxygenase-2 mediated through TOLL-like receptor 4,” Journal of Biological Chemistry, vol. 276, pp. 16683-16689 (2001). Briefly, the luciferase reporter plasmids (pGL2) containing the promoter region of the murine COX-2 gene (−3201/+93 or −1017/+93) were provided by David Dewitt (Michigan State University, East Lansing, Mich.). To prepare the wild-type COX-2 promoter fragment, polymerase chain reaction (PCR) was performed with the primers named Kpn-COX2-For and Hind-COX2-Rev using the murine COX-2 (−1017/+93) luciferase reporter plasmid as a template. To prepare the mutant COX-2 promoter fragment containing mutated NFκB site, Kpn-COX2-Fmut and Hind-COX2-Rev were used as primers. Each PCR fragment was inserted into the KpnI and HindIII sites of pGL2 to generate the wild-type or mutant COX-2 (−410/+86) luciferase reporter constructs, respectively. Sequences for wild-type NFκB site and the primers were used as reported elsewhere. (See Lee et al., 2001). The 2×NFκB-luciferase reporter construct was a gift from Frank Mercurio (Signal Pharmaceuticals, San Diego, Calif.). HSP70-β-galactosidase reporter plasmid was a gift from Robert Modlin (University of California, Los Angeles, Calif.). The expression plasmids for a constitutively active form of TLR4 (DTLR4) and a dominant-negative mutant, DTLR4 (P712H) were prepared as previously described. See S. H. Rhee et al., 2000. The expression plasmid of the wild-type NFκB-inducing kinase (NIK), pRK-NIK(wt) was a gift from Mike Rothe (Tularik, South San Francisco, Calif.). The dominant-negative mutant of inhibitor kB (pCMV4-IkBa(DN)) was provided by Dean Ballard (Vanderbilt University, Nashville, Tenn.). 4×NFκB-luciferase reporter construct was purchased from Clontech (Palo Alto, Calif.) and used for transient transfection. Heat shock protein 70 (HSP70)-β-galactosidase reporter plasmid was from Robert Modlin (University of Calif., Los Angeles, Calif.). The expression plasmids for a wild-type TLR2 and a dominant-negative mutant [TLR2 (P681H)] were from C. B. Wilson (University of Washington, Seattle, Wash.). Constitutively active chimeric CD4-TLR4 was obtained from C. A. Janeway, Jr. (Yale University, New Haven, Conn.). The constitutively active form of MyD88 (MyD88(ΔToll)) and the dominant-negative mutant, MyD88(ΔDD) were kindly provided by Jurg Tschopp (University of Lausanne, Switzerland). The dominant-negative mutant of mouse PPARg [pCMX-PPARg(L466A/L467A)] was a gift from Ira Schulman (Ligand Pharmaceuticals, San Diego, Calif.). All DNA constructs were prepared in large scale using the EndoFree Plasmid Maxi kit (Qiagen, Chatsworth, Calif.) for transfection.

Preparation of stably transfected cells with luciferase reporter plasmids. RAW 264.7 cells (1×10⁶ cells) were plated in 100 mm dish and transfected with murine COX-2 promoter (−3.2 kb) luciferase plasmid using Superfect Transfection reagent (Qiagen) according to the manufacturer's instruction. pcDNA3/neo was co-transfected to select transfected cells using the antibiotic. After 48 hrs of stabilization, the new media containing Geneticin (500 μg/ml) was added and changed for appropriate time periods. Two weeks later, the colonies that survived were selected and propagated under Geneticin. After another two weeks of antibiotic selection, the luciferase activities were determined for each colony after treatment with LPS (100 ng/ml). The colony that showed the highest response to LPS treatment was selected.

Transient transfection and luciferase assay—These procedures were performed as described in S. H. Rhee et al., “Murine TOLL-like receptor 4 confers lipopolysaccharide responsiveness as determined by activation of NFκB and expression of the inducible cyclooxygenase,” J. Biol. Chem., vol. 275, pp. 34035-34040 (2000); and J. H. Paik et al., “Two opposing effects of non-steroidal anti-inflammatory drugs on the expression of the inducible cyclooxygenase, Mediation through different signaling pathways,” J. Biol. Chem., vol. 275, pp. 28173-28179 (2000). Briefly, RAW 264.7 cells were plated in 6-well plates (5×10⁵ cells/well) and transfected with luciferase reporter plasmids and HSP70-β-galactosidase plasmid as an internal control using SuperFect transfect reagent (Quiagen) according to the manufacturer's instructions. Luciferase and β-galactosidase enzyme activities were determined using the Luciferase Assay System and β-galactosidase Enzyme System (Promega, Madison, Wis.) according to the manufacturer's instructions. Luciferase activity was normalized by β-galactosidase activity.

Human studies to determine whether dietary n-3 polyunsaturated fatty acids (PUFAs) suppress the expression of COX-2 induced by TLR4 agonist (lipopolysaccharide) in peripheral blood monocytes. Two randomized, double-blind, placebo-controlled, parallel arm studies were conducted in human subjects where the amounts of dietary n-3 and n-6 PUFAs were controlled using institutionally prepared diets with fish oil concentrate or placebo oil capsules. The study protocol was approved by the local Institutional Review Board, and subjects gave written informed consent. In Study I, subjects (7 to 8 per group) received 9 g of purified fish oil with varying amounts of linoleic acid (LA, C18:2n-6) for 8 weeks, whereas in Study II, subjects (11 to 12 per group) received varying amount of fish oil (0,6, 15 g) with a constant amount of LA for 4 weeks. The control group received LA without fish oil supplementation. Details for the study design and provision of the diets are described in D. Hwang et al., “Does vegetable oil attenuate the beneficial effects to fish oil reducing risk factors for cardiovascular disease?”, Am. J. Clin. Nutr., vol. 66, pp. 89-96 (1997).

Assay of cyclooxygenase-2 (COX-2) activity in human monocytes. Briefly, monocytes isolated from peripheral blood as described in S. J. McFaul, “A method for isolating neutrophils from moderate volumes of human blood,” J. Immunol. Methods, vol. 130, pp. 15-18 (1990). The isolated monocytes were pretreated with aspirin to inactivate any endogenous COX. Newly expressed COX in response to lipopolysaccharide (LPS, 100 ng/ml) stimulation was determined by measuring the production of PGE₂ in the presence of arachidonic acid (AA; 30 μM, above Vmax). Only COX-2, but not COX-1, is selectively expressed in LPS-stimulated macrophages. See S. H. Lee et al., “Selective expression of mitogen-inducible cyclooxygenase in macrophages stimulated with lipopolysaccharide,” J. Biol. Chem., vol. 267, pp. 25934-25938 (1992). Therefore, COX activity measured at these conditions reflects that of de novo synthesized COX-2.

Statistical analysis. Data were analyzed by a paired t-test with a significance level of p <0.05.

### EXAMPLE 2

### Saturated Fatty Acids, but not Unsaturated fatty Acids, Induced COX-2 Expression in RAW 264.7 cells

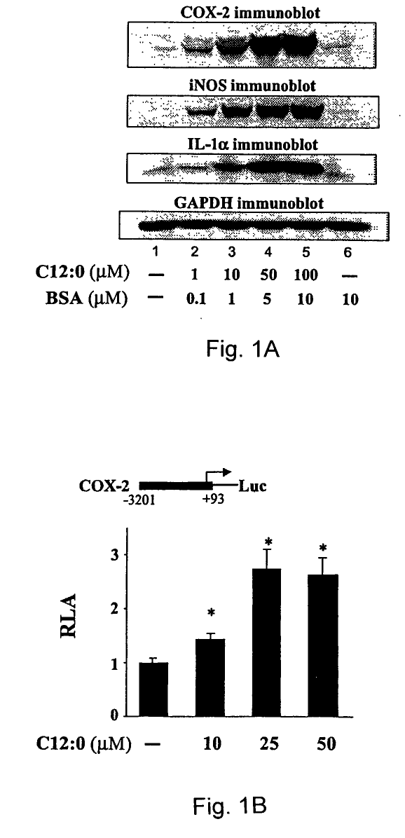

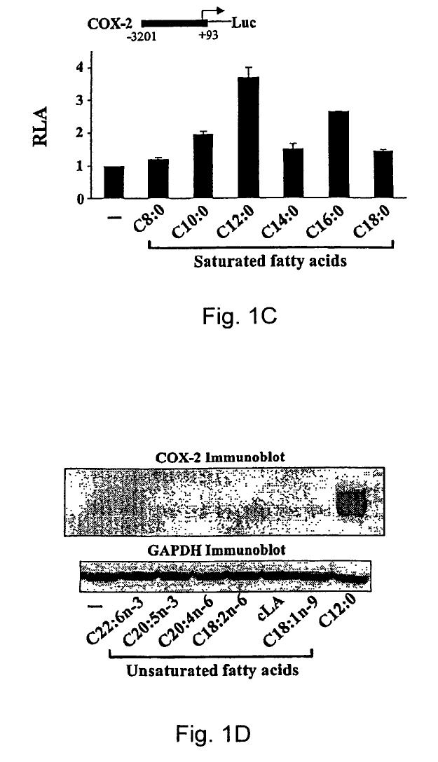

RAW 264.7 cells were maintained in serum-poor (0.25%) medium, and were treated with various concentrations (1, 10, 50, and 100 μM) of lauric acid (C12:0) solubilized with BSA at a molar ratio of 10:1 (fatty acid:BSA). After 11 h, cell lysates were analyzed by COX-2, iNOS, IL-1α, or GAPDH immunoblot using a Western blot analysis. The results are shown in FIG. 1A. Lane 1 indicates cells treated in medium alone; lanes 2-5 represent cells treated with lauric acid in medium with BSA; and lane 6 represents cells treated in medium with 10 μM BSA without fatty acid. As shown in FIG. 1A, the saturated fatty acid lauric acid induced COX-2, iNOS, and IL-1α expression.

RAW 264.7 cells were transfected with a luciferase reporter for the COX-2 promoter and the HSP70-α-galactosidase reporter as an internal control. These transfected cells were then treated for 24 hr with either various concentrations of lauric acid, including 10, 25, and 50 μM; or with 75 μM of each of several saturated fatty acids. The luciferase and α-galactosidase enzyme activities were measured as described in Example 1. Relative luciferase activity was determined by normalization with α-galactosidase activity. As shown in FIG. 1B, lauric acid induced COX-2 expression as measured using the luciferase reporter gene assay. In FIGS. 1B and 1C, the panels contain representative data from more than three different experiments. Values are mean±standard error (n=3). An asterisk * in FIG. 1B indicates a value significantly different from the vehicle control.

Among the saturated fatty acids tested (C8:0-C18:0), lauric acid (C12:0) and palmitic acid (C16:0) were most potent in inducing COX-2 expression (FIG. 1C).

RAW 264.7 cells were then treated with 75 μM of lauric acid and various unsaturated fatty acids, including docosahexaenoic acid (DHA; C22:6n-3), eicosapentaenoic acid (EPA; C20:5-n3), arachidonic acid (AA; C-20:4n-6), linoleic acid (LA; C18:2n-6), conjugated linoleic acid (cLA), and oleic acid (OA; C18:1n-9). The cell lysates were analyzed by COX-2 or GAPDH immunoblot. Unlike saturated fatty acids, all unsaturated fatty acids tested (C18:1n-9, C18:2n-6, C20:4n-6, C20:5n-3, C22:6n-3, and cLA) were unable to induce COX-2 expression in RAW 264.7 cells (FIG. 1d).

All saturated fatty acids tested induced COX-2 expression, while all unsaturated fatty acids tested (including n-3, n-6 and n-9) were unable to induce COX-2 expression.

### EXAMPLE 3

### Induction of COX-2 Expression by Saturated Fatty Acids is Mediated through the Activation of NFκB

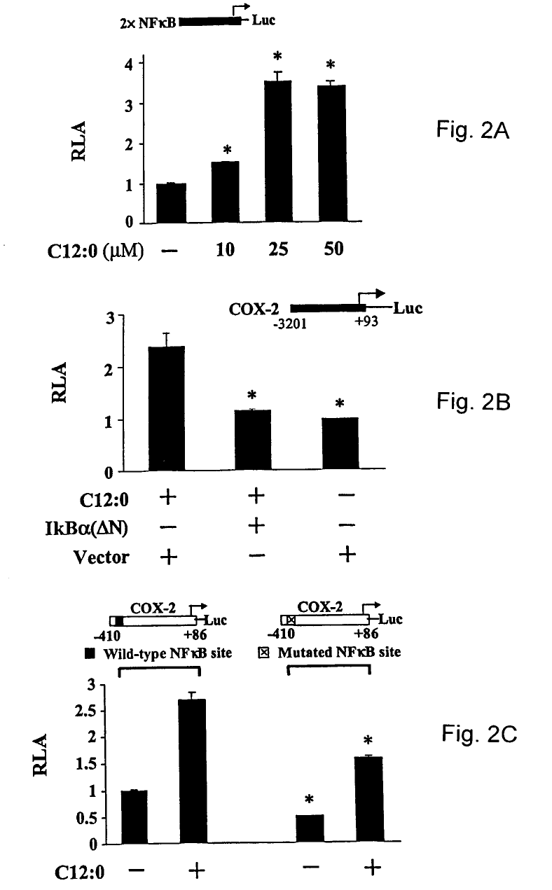

In previous studies we demonstrated that activation of NFκB is sufficient and required to induce COX-2 expression in RAW 264.7 cells. See Rhee et al., 2000. Therefore, an experiment was run to determine whether saturated fatty acid-induced COX-2 expression is mediated through the activation of NFκB in RAW 264.7 cells. RAW 264.7 cells were transfected with a luciferase (Luc) reporter plasmid for NFκB response element and treated with various concentrations (10, 25, and 50 μM) of lauric acid for 24 h. The relative luciferase activity was determined as described in Examples 1 and 2. As shown in FIG. 2A, lauric acid activated NFκB in a dose-dependent manner. The panels are representative data from more than three different experiments. Values are mean±SEM (n=3). An asterisk * means that the panel is significantly different from the vehicle control, p<0.05.

To further show the role of NFκB, RAW 264.7 cells were co-transfected with a luciferase reporter plasmid for COX-2 promoter and the expression plasmid containing a dominant-negative mutant of IkBa [IkBa(DN)], and then treated with 75 mM of lauric acid (C12:0) for 24 hours. The expression of COX-2 induced by lauric acid was inhibited by co-transfection of the dominant-negative mutant of IkBa plasmid (FIG. 2B). The panels are representative data from more than three different experiments. Values are mean±SEM (n=3). An asterisk * means that the panel is significantly different from the control (C12:0+vector),p<0.05.

In addition, RAW 264.7 cells were transfected with a luciferase reporter plasmid for COX-2 promoter containing wild-type NFkB site or mutated NFkB site. Relative luciferase activity (RLA) was determined as described in Example 2. The panels are representative data from more than three different experiments. Values are mean±SEM (n=3). An asterisk * means that the panel is significantly different from the data obtained using COX-2 promoter with wild-type NFkB site, p<0.05. As shown in FIG. 2C, lauric acid-induced COX-2 expression was significantly reduced in the COX-2 promoter reporter gene containing mutated NFκB site as compared with the one containing wild-type NFκB site.

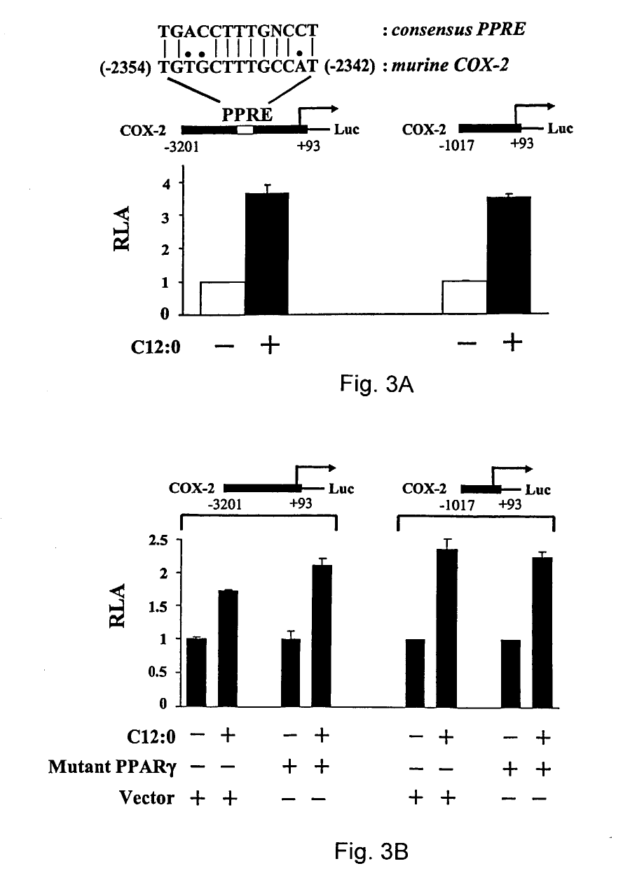

Since naturally occurring fatty acids are known to bind and activate PPARs, and some PPAR activators induce COX-2 expression in certain cell types, an experiment was run to determine whether saturated fatty acid-induced COX-2 expression is also mediated through PPAR signaling pathway. The 5′-flanking region of murine COX-2 contains PPAR response element (PPRE)-like sequences, as shown in FIG. 3A. The experiment was designed to determine whether these sequences are required for saturated fatty acid-induced COX-2 expression. RAW 264.7 cells were transfected with a luciferase reporter plasmid for COX-2 promoter with or without PPRE-like sequences. The results showed that deletion of those sequences did not affect the promoter activity of COX-2 reporter gene (FIG. 3A). Relative luciferase activity (RLA) was determined as described in Example 2. The panels are representative data from more than three different experiments. Values are mean±SEM (n=3).

Next, I determined whether a dominant-negative mutant of PPARγ altered saturated fatty acid-induced COX-2 expression. RAW 264.7 cells were co-transfected with a reporter plasmid for COX-2 promoter, with or without a dominant-negative mutant of PPARγ, and then treated with lauric acid (75 mM) for 24 hours. Relative luciferase activity (RLA) was determined as described in Example 2. The results showed that lauric acid-induced COX-2 expression in cells co-transfected with a dominant-negative mutant of PPARγ plasmid was not altered as compared with control cells, regardless of whether the COX-2 reporter gene construct contained the PPRE-like sequences or not (FIG. 3B). The panels are representative data from more than three different experiments. Values are mean±SEM (n=3).

Together, these results suggest that saturated fatty acid-induced COX-2 expression is not directly mediated through the PPRE-like sequences in COX-2 gene.

### EXAMPLE 4

### Saturated Fatty Acid-induced COX-2 Expression was Inhibited by a Dominant-negative Mutant of TLR4

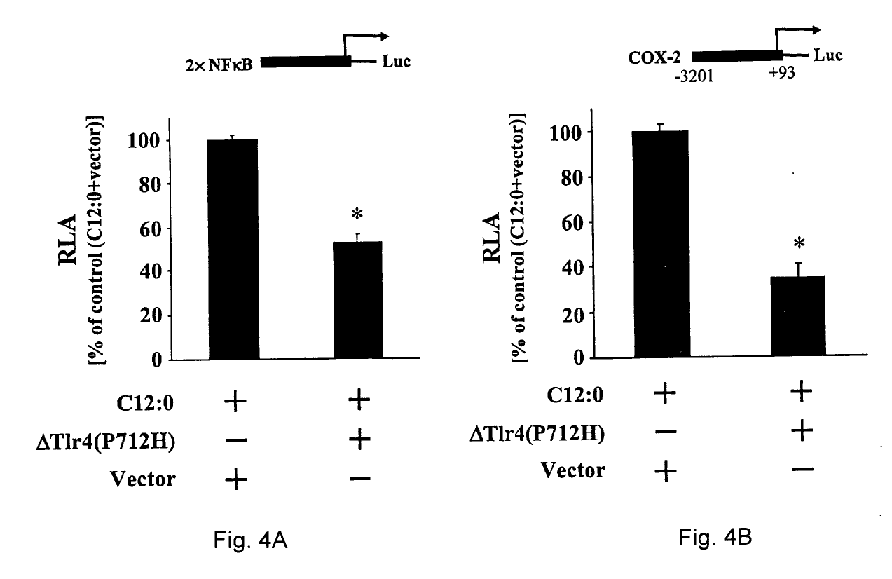

Next, an attempt was made to identify the upstream target in the NFκB signaling pathways through which the saturated fatty acids activate NFκB and induce COX-2 expression. An experiment was run to determine whether saturated fatty acid-induced activation of NFκB and COX2 expression are mediated through the murine LPS receptor (TLR4). If saturated fatty acid-induced COX-2 expression is mediated through TLR4, co-transfection of cells with a dominant-negative mutant of TLR4 should lead to inhibition of COX-2 expression. RAW 264.7 cells were cotransfected with a luciferase reporter plasmid for NFκB response element (FIG. 4A) or COX-2 promoter (FIG. 4B) and the expression plasmid for a dominant-negative mutant of TLR4 [DTLR4 (P712H)], and were then treated with lauric acid (75 mM) for 24 hours. Relative luciferase activity (RLA) was determined as described in Example 2. The panels are representative data from more than three different experiments. Values are mean±SEM (n=3). An asterisk * indicates significantly different from the control (C12:0+vector), p<0.05. The results show that the dominant-negative mutant of TLR4 [DTLR4 (P712H)] inhibits both saturated fatty acid-induced NFκB activation and COX-2 expression (FIGS. 4A and 4B). These results suggest that the upstream target in the signaling pathways through which saturated fatty acids mediate NFκB activation and COX-2 expression is TLR4.

### EXAMPLE 5

### Unsaturated Fatty Acids Inhibited Saturated Fatty Acid-induced COX-2 Expression, and this Inhibition was Mediated Through Suppression of NFκB

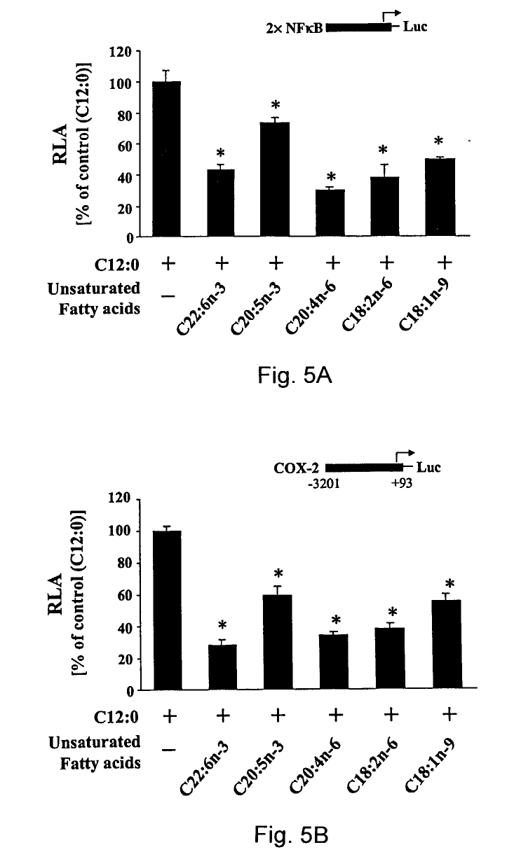

Unlike saturated fatty acids, unsaturated fatty acids were unable to induce COX-2 expression (FIG. 1D). To further determine the effects of unsaturated fatty acids, RAW 264.7 cells were transfected with a luciferase reporter plasmid for NFκB response element (FIG. 5A) or COX-2 promoter (FIG. 5B) and pre-treated with 5 mM of each unsaturated fatty acid for 3 hours, and then treated with lauric acid (75 mM) for additional 21 hours. Relative luciferase activity (RLA) was determined as described in Example 2 and data are expressed as a percentage of the lauric acid control (C12:0). The panels are representative data from more than three different experiments. Values are mean±SEM (n=3). An asterisk * indicates significantly different from the control C12:0 alone,p<0.05. The results indicated that unsaturated fatty acids inhibited saturated fatty acid-induced NFκB activation (FIG. 5A) and COX-2 expression (FIG. 5B). These results indicate that inhibition of saturated fatty acid-induced COX-2 expression by unsaturated fatty acids is mediated through suppression of NFκB signaling pathway. Together, these results suggest that both the induction of COX-2 by saturated fatty acids and its inhibition by unsaturated fatty acids are mediated through the NFκB signaling pathway.

### EXAMPLE 6

### Unsaturated Fatty Acids Inhibited Constitutively Active TLR4 (DTLR4)-induced COX-2 Expression, but did not Inhibit COX-2 Expression Induced by Constitutively Active MyD88 or NFκB-inducing Kinase (NIK) which lies Downstream of TLR4

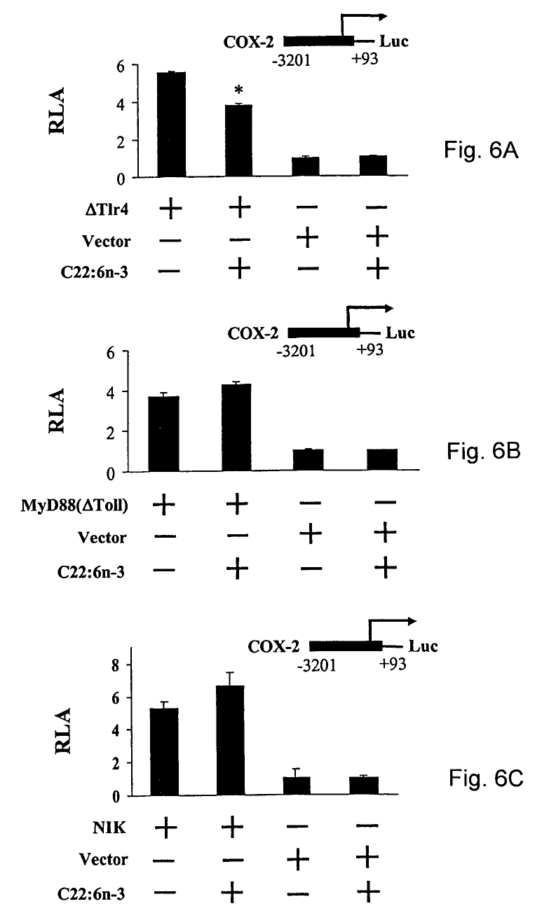

If saturated fatty acid-induced COX-2 expression is mediated through TLR4, then it is logical to determine whether the inhibition of saturated fatty acid-induced COX-2 expression by unsaturated fatty acids is also mediated through TLR4. RAW 264.7 cells were co-transfected with a luciferase reporter plasmid for COX-2 promoter and the expression plasmid for a constitutively active TLR4 (DTLR4) (FIG. 6A), a constitutively active MyD88(DToll) (FIG. 6B), or NIK (FIG. 6C), and then treated with 20 mM of docosahexaenoic acid (C22:6n-3) for 11 hours. Relative luciferase activity (RLA) was determined as described in Example 2. The panels are representative data from more than three different experiments. Values are mean±SEM (n=3). An asterisk * indicates significantly different from the control (DTLR4 without C22:6n-3), p<0.05.

The results showed that docosahexaenoic acid (C22:6n-3) partially inhibited constitutively active TLR4 (DTLR4)-induced COX-2 expression (FIG. 6A). MyD88 is the immediate downstream adaptor protein which interacts directly with the cytoplasmic domain of TLR4. Activation of MyD88 leads to activation of NFκB and COX-2 expression in RAW 264.7 cells. See Rhee et al., 2000. Therefore, if the inhibition of saturated fatty acid-induced COX-2 expression by unsaturated fatty acids is mediated through TLR4, COX-2 expression induced by the activation of signaling steps downstream of TLR4 should not be inhibited by unsaturated fatty acids. The results indeed showed that docosahexaenoic acid (C22:6n-3) was unable to inhibit COX-2 expression induced by constitutively active MyD88 or NIK (FIG. 6B and 6c). These results suggest that both induction of COX-2 expression by saturated fatty acids and its inhibition by unsaturated fatty acids are mediated through a common upstream signaling step, that is, TLR4.

### EXAMPLE 1

### Unsaturated Fatty Acid also Inhibited LPS-induced NFκB Activation and Expression of COX-2, iNOS and IL-1α

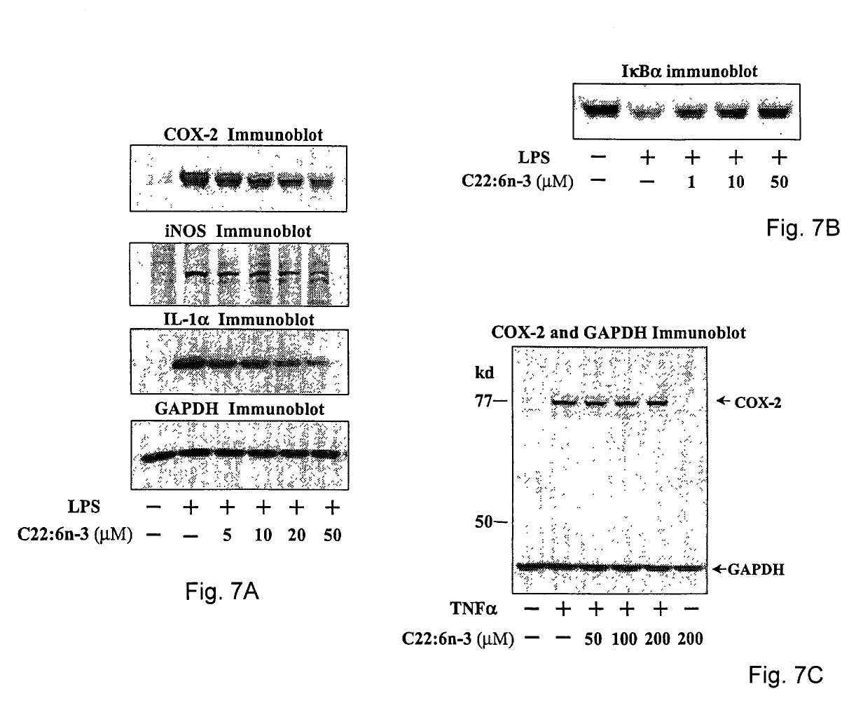

If the inhibition of saturated fatty acid-induced COX-2 expression by unsaturated fatty acids is mediated through TLR4, then unsaturated fatty acids should also inhibit LPS-induced COX2 expression. RAW 264.7 cells were pretested with various concentrations (5, 10, 20, and 50 μM) of docosahexaenoic acid (C22:6n-3) for 3 hours, and then stimulated with LPS (100 ng/ml) for 8 hours and analyzed by COX-2, iNOS, IL-1 a or GAPDH immunoblot. The results are shown in FIG. 7A. The results indeed show that docosahexaenoic acid (C22:6n-3) inhibits the LPS-induced expression of COX-2, iNOS, and IL-1 a (FIG. 7A). Other unsaturated fatty acids tested, as used in Example 2 and FIG. 1D, also inhibited LPS-induced COX-2 expression (data not shown).

RAW 264.7 cells were pretested with various concentrations (5, 10,20, and 50 μM) of docosahexaenoic acid (C22:6n-3) for 3 hours, and then stimulated with LPS (100 ng/ml) for 30 min and analyzed by IkBa immunoblot. The results are shown in FIG. 7B. Inhibition of LPS-induced NFκB activation by docosahexaenoic acid (C22:6n-3) is demonstrated by inhibition of LPS-induced degradation of IkBa protein (FIG. 7B).

Colon cancer cells (HT-29) were pretested with various concentrations of docosahexaenoic acid (C22:6n-3) for 3 hours and then treated with TNFα (20 ng/ml) for 8 hours. Cell lysates were analyzed by COX-2 and GAPDH immunoblot. The results are shown in FIG. 7C. Docosahexaenoic acid (C22:6n-3) failed to inhibit TNFα-induced COX-2 expression in a colon tumor cell line (HT-29) (FIG. 7C), reinforcing the possibility that the inhibitory effect of unsaturated fatty acid on saturated fatty acid- or LPS-induced expression of COX-2 is specifically mediated through TLR4.

### EXAMPLE 8

### In Vivo Experiments in Mice

Varying doses of unsaturated fatty acids, essentially free of saturated fatty acids, will be infused into mice that have been injected with a lethal dose of LPS (1.5 mg/mouse). The unsaturated fatty acids will be given in different trials both as a single unsaturated fatty acid and as a mixture of two or more unsaturated fatty acids. The mortality of the mice will be monitored for one week. Blood levels of TNFα will be monitored as described by A. Novogrodsky et al., “Prevention of lipopolysaccharide-induced lethal toxicity by tyrosine kinase inhibitors,” Science, vol. 264, pp. 1319-1322 (1994).

In a second set of mice experiments, the mice will be infused with varying doses of a triglyceride which contains only unsaturated fatty acids. The rest of the experiment will be as discussed above.

Once satisfactory data from laboratory animals have been obtained, clinical trials in human patients will be conducted in accordance with applicable statutes and regulations.

The above examples have shown that saturated fatty acids, but not unsaturated fatty acids, induce COX-2 in RAW 264.7 cells (FIGS. 1A, 1B, 1C, and 1D). Greater potency of lauric acid and palmitic acid in inducing COX-2 expression among saturated fatty acids tested (FIG. 1C) coincides with the abundance of these fatty acids in lipid A molecule. Lauric acid, myristic and palmitic acid are known to be major fatty acids acylated in lipid A molecule of LPS. The fact that deacylation of these fatty acids from LPS results in loss of endotoxic activity implies an important role of these fatty acids in LPS-mediated signal transmission. NFκB is one of the major downstream signaling pathways derived from activation of LPS receptor, TLR4 in RAW 264.7 cells. The results demonstrating that induction of COX-2 by lauric acid is mediated through activation of NFκB (FIG. 2) and that this activation is inhibited by a dominant-negative mutant of TLR4 (FIG. 4A), suggest that the most upstream signaling component affected by saturated fatty acids is TLR4 or molecules associated with either extracellular or intracellular domains of TLR4.

The results presented in FIGS. 4A, 4B, 6A, 6B, and 6C suggest that activation Of NFκB and COX-2 expression induced by saturated fatty acids and inhibition of this induction by unsaturated fatty acids are mediated through a common upstream signaling step, TLR4.

### EXAMPLE 9

### Effects of Dietary n-3 Polyunsaturated Fatty Acids on LPS-stimulated COX-2 Expression in Human Monocytes

To determine whether the intake of fish oil, a major dietary source of docosahexaenoic acid (DHA, C22:6n-3) and eicosapentaenoic acid (EPA, C20:5n-3), lead to suppression of COX-2 expression in human monocytes exposed to LPS in vitro, humans were fed various diets as described above in Example 1. Two randomized, double-blinded, placebo controlled, parallel arm studies were conducted in healthy subjects where the amounts of dietary n-3 and n-6 polyunsaturated fatty acids (PUFAs) were controlled using institutionally prepared diets and fish oil concentrate or placebo oil capsules. Fish oil concentrate or safflower oil was used as a source of n-3 PUFAs or n-6 PUFAs (linoleic acid), respectively. In study I, subjects consumed an equal amounts of fish oil with varying amounts of the n-6 fatty acid. In study II, subjects consumed various amounts of n-3 PUFAs with a constant amount of n-6 PUFAs. Peripheral blood monocytes were isolated, and lipopolysaccharide (LPS)-induced COX-2 expression in monocytes was determined as described in Example 1. COX-2 expression was significantly (p<0.022) suppressed by high doses (15 g/day) of fish oil, but not by the lower doses (6 g/day and 9 g/day). (Data not shown).

These results indicate that the suppression of COX-2 and cytokine expression by fish oil intake occurs at high dose levels. Since the feeding periods were relatively short (4-8 weeks), it is possible that the suppression of the expression of COX-2 and cytokines could occur at lower levels of fish oil intake if the experimental period were prolonged.

### EXAMPLE 10

### Effect of n-3 PUFAs of LPS-Induced NFκB Activation and COX-2 Expression

To investigate the mechanism by which n-3 PUFAs inhibit LPS-induced COX-2 expression in human blood monocytes, the relative potency of unsaturated fatty acids was determined in inhibiting the TLR-induced signaling pathway and the target gene expression in a murine monocytic cell line (RAW 264.7) which were stably transfected with either NFκB or COX-2 promoter luciferase reporter gene. These stably transfected cell lines eliminated the necessity of transfecting plasmids for the reporter gene and the internal control. Thus, inhibitory or stimulatory effects of various fatty acids on agonist-induced TLR activation were quantitatively determined in a high throughput mode using 96 well plates. Demonstration that LPS-induced NFκB activation and COX-2 expression in RAW 264.7 cells is mediated through TLR4 was reported above in Examples 5-7. Therefore, NFκB activation and COX-2 expression was used as readouts for agonist-induced TLR activation, and its suppression or potentiation by fatty acids in this experiment.

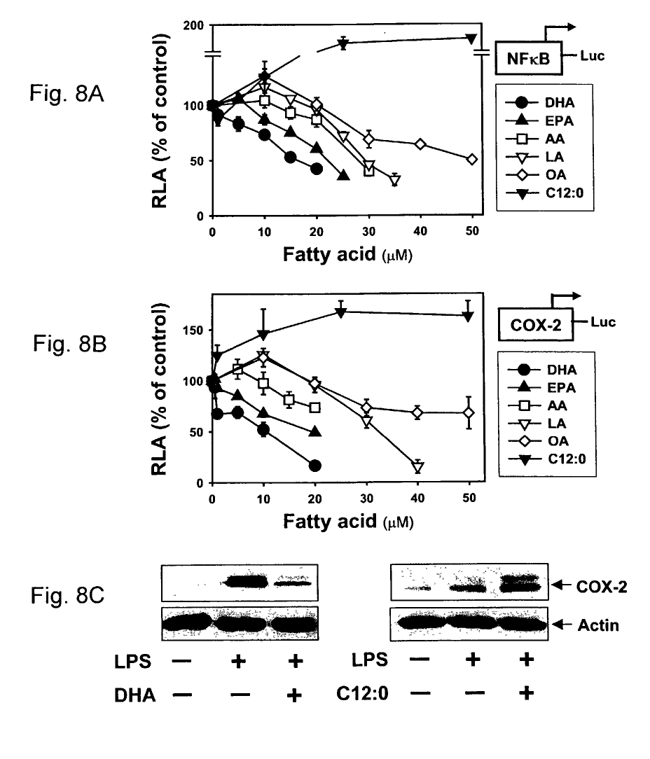

Cells stably transfected with NFκB(5×) or COX-2 promoter reporter genes were pretreated with various concentrations of each fatty acid for 3 hrs. Cells were then treated with LPS (200 ng/ml). After 8 hrs, cell lysates were prepared and luciferase activities were determined. Data are expressed as a percentage of LPS treatment alone. The results for NFκB and COX-2 expression are given in FIGS. 8A and 8B, respectively. The values are mean±SEM (n=3) of RLA, relative luciferase activity. In FIG. 8C, the cells were pretreated with DHA (20 μM) or C12:0 (75 μM) for 3 hrs and further stimulated with LPS (200 ng/ml for DHA; 1 ng/ml for C12:0). After 8 hr, cell lysates were analyzed by COX-2 and actin immunoblotting. In FIG. 8A, 8B, and 8C, the following abbreviations are used: DHA, docosahexaenoic acid (C22:6n-3); EPA, eicosapentaenoic acid (C20:5n-3); AA, arachidonic acid (C20:4n-6); LA, linoleic acid (C18:2n-6); OA, oleic acid (C18:1n-9); and C12:0, lauric acid.

All unsaturated fatty acids tested inhibit ed LPS-induced NFκB activation and COX-2 expression as determined by reporter gene assays (FIGS. 8A and 8B). Among unsaturated fatty acids, DHA and EPA were the most potent inhibitors. This finding corroborated the results from the human studies described above in Example 9, and demonstrated that n-3 PUFAs (DHA and EPA) as compared with n-6 PUFAs (AA and LA) are much more potent inhibitors of TLR4 activation. In contrast, a saturated fatty acid, lauric acid (C12:0), potentiated LPS-induced NFκB activation and COX-2 expression (FIGS. 8A and 8B). Results from Examples 2-4 above showed that saturated fatty acids alone, without other agonists, can induce NFκB activation and COX-2 expression in RAW 264.7 cells. Immunoblot analyses also showed that LPS-induced COX-2 expression is suppressed by DHA but potentiated by the saturated fatty acid (FIG. 8C).

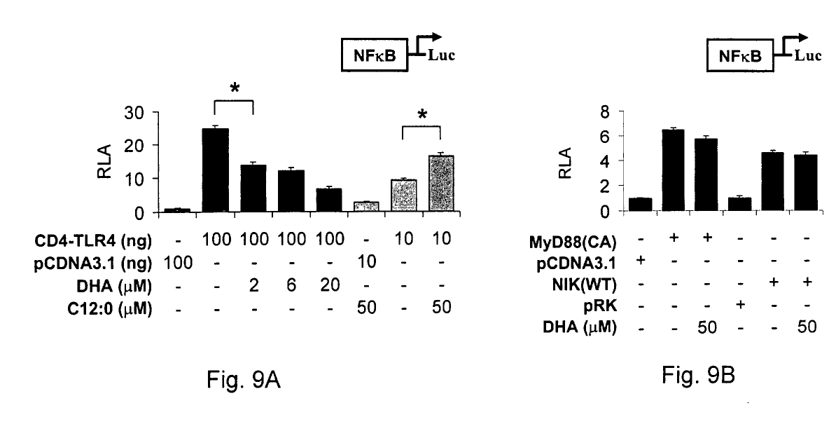

To determine whether unsaturated fatty acids inhibit the activation of TLR4 in a reconstituted system, human embryonic kidney cells (293T) were transfected with a constitutively active form of TLR4 (CD4-TLR4) to activate TLR4-mediated signaling pathways in a ligand-independent manner. Cells were co-transfected with NFκB-luciferase reporter plasmid and the expression plasmid of constitutively active human TLR4 (CD4-TLR4), and treated with DHA or C12:0. The results are shown in FIG. 9A. Cells were also co-transfected with NFκB-luciferase reporter plasmid and the expression plasmid of constitutively active MyD88 [MyD88(CA)] or wild type NIK [NIK(WT)], and treated with DHA. The results are shown in FIG. 9B. In both FIGS. 9A and 9B, the values are mean±SEM (n=3), RLA, relative luciferase activity. An “*” indicates a significant difference from the respective control (p<0.05).

As shown in FIG. 9A, DHA inhibited, but C12:0 potentiated CD4-TLR4-induced NFκB activation in 293T cells. These results are consistent with the results demonstrating the similar pattern of modulation by fatty acids for the ligand-induced activation of TLR4 in RAW 264.7 cells (FIG. 8A).

To determine whether the target of inhibition by DHA is TLR4 or its downstream signaling components, a known common component of the immediate downstream signaling pathways of all TLRs, the adaptor protein, myeloid differential factor (MyD88), was used. The activation of NFκB mediated through MyD88 is known to be one of the major downstream signaling pathways derived from TLRs. As shown in FIG. 9B, DHA does not inhibit NFκB activation induced by the activation of downstream component (MyD88 or NIK) of TLR signaling pathways. These results are in contrast to the inhibitory effect of unsaturated fatty acids on both NFκB activation induced by both TLR4 agonist (LPS) (FIG. 8A) and constitutively active TLR4 (CD4-TLR4) (FIG. 9A). These results suggest that the molecular target of inhibition by DHA is TLR itself or its associated molecules but not the downstream components.

### EXAMPLE 11

### Effect of n-3 PUFAs and Saturated Fatty Acids on TLR2 Agonist (PamCAG)-induced NFκB Activation and COX-2 Expression

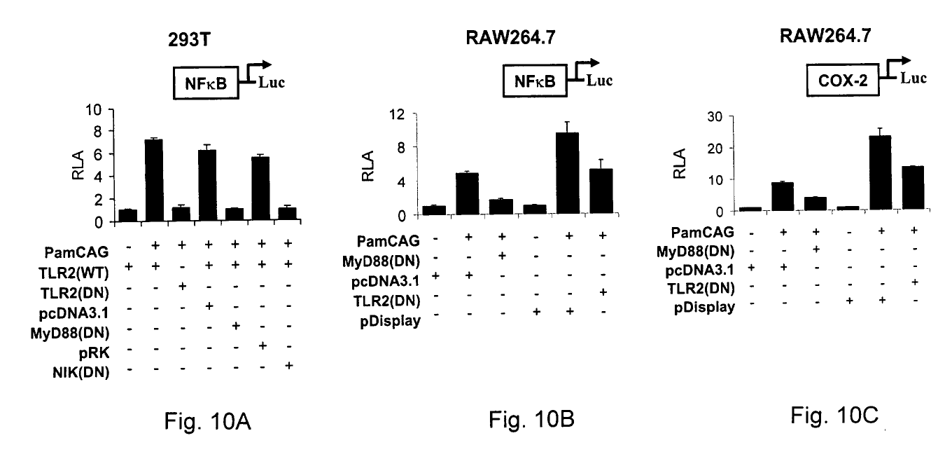

Acylation by saturated fatty acids of bacterial lipopeptides is also required for the activation of TLR2. An experiment was conducted to determine whether unsaturated fatty acids suppress the activation of TLR2 as they do TLR4 activation. Initially an experiment was conducted to demonstrate that PamCAG, a synthetic analog of bacterial lipopeptides which are known agonists of TLR2, activated TLR2 in a reconstituted system using 293T cells which do not express TLR2. The results are shown in FIGS. 10A, 10B, and 10C. Either 293T (FIG. 10A) or RAW 264.7 cells (FIGS. 10B and 10C) were transfected with NFκB (FIGS. 10A and 10B) or COX-2 (FIG. 10C) luciferase reporter plasmid and co-transfected with various expression plasmids as indicated on the figures. pcDNA3.1, pRK, and pDisplay were transfected as vector controls for the dominant negative (DN) mutants of MyD88, NIK, and TLR2, respectively. Twenty-four hours after the transfection, cells were stimulated with either vehicle control or PamCAG (1 μg/ml). After 18 hr, luciferase activities were determined. Values shown are mean±SEM (n=3). The abbreviations used in the figure are the following: WT, wild type; and RLA, relative luciferase activity.

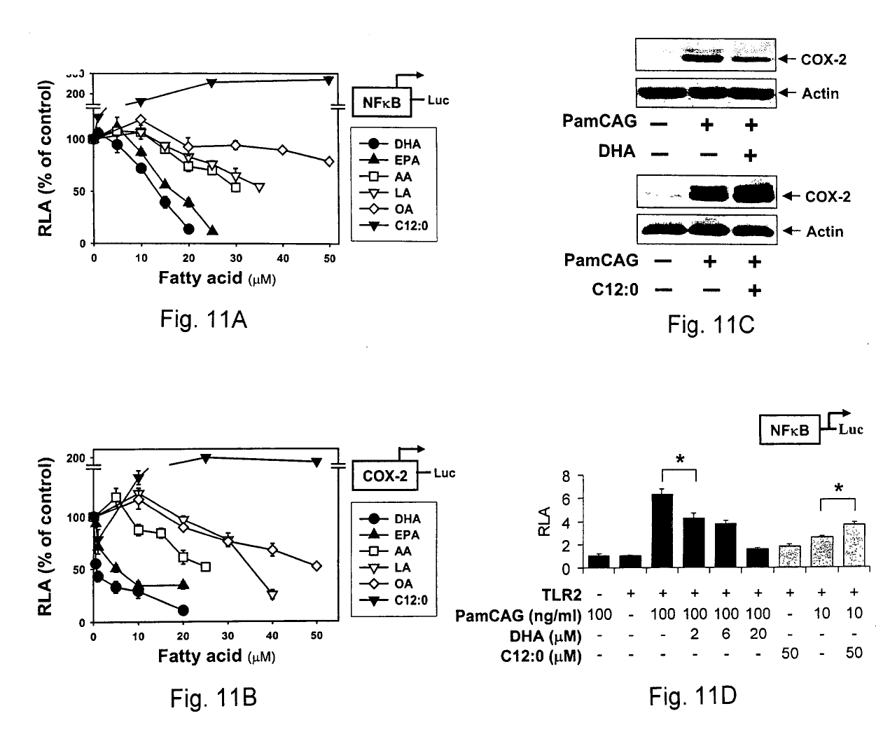

PamCAG activated TLR2, as determined by NFκB activation and by its inhibition by either a dominant negative mutant of TLR2 or a downstream signaling component (MyD88 or NIK) in TLR2-transfected 293T cells. (FIG. 10A). The murine monocytic cell line (RAW 264.7) expresses both TLR2 and TLR4. RAW 264.7 cells were stably transfected with NFκB(5×) (FIG. 11A) or COX-2 (FIG. 11B) promoter reporter gene and were pretreated with various concentrations of each fatty acid for 3 hrs. Cells were further stimulated with a synthetic bacterial lipopepetide (PamCAG, 500 ng/ml). After 8 hrs, luciferase activities were determined. The results are shown in FIGS. 11A and 11B. Data are expressed as a percentage of PamCAG treatment alone.

RAW 264.7 cells were then pretreated with DHA (20 μM) or C12:0 (75 μM) for 3 hrs and further stimulated with PamCAG (500 ng/ml). After 8 hrs, cell lysates were analyzed by COX-2 and actin immunoblotting. The results are shown in FIG. 11C. 293T cells were cotransfected with NFκB-luciferase reporter plasmid and TLR2 expression plasmid, and treated with PamCAG (100 ng/ml) in the presence or absence of DHA or C12:0. The results are shown in FIG. 11D. In FIG. 11D, values are mean±SEM (n=3). An “*” indicates a significant difference from the respective control (p<0.05).

FIGS. 10B and 10C indicate that PamCAG activated endogenous TLR2 in RAW 264.7 cells. PamCAG induced NFκB activation and expression of COX-2, and this induction was inhibited by a dominant negative mutant of TLR2 or MyD88 (FIGS. 10B and 10C). These results demonstrated that PamCAG activates both ectopically expressed TLR2 in 293T cells and endogenous TLR2 in RAW 264.7 cells.

Similar to the results shown with TLR4 agonist (FIGS. 8A, 8B, and 8C), n-3 PUFAs, DHA and EPA, were the most potent inhibitors among the unsaturated fatty acids tested for PamCAG-induced NFκB activation and COX-2 expression in RAW 264.7 cells (FIGS. 11A, 11B and 11C). The saturated fatty acid (C12:0) potentiated PamCAG-induced NFκB activation and COX-2 expression in RAW 264.7 cells. In addition, DHA inhibited, but lauric acid (C12:0) potentiated NFκB activation induced by PamCAG in TLR2-transfected 293T cells (FIG. 11D).

These results demonstrate the modulatory effects of the fatty acids on the activation of both TLR2 and TLR4; i.e., saturated fatty acids stimulate and unsaturated fatty acids inhibit the activation. The results also indicate that the molecular target for the inhibition is either the Toll-receptor or an associated molecule but not the downstream pathway. Since fatty acids modulated TLR2 also, use of fatty acids will also be effective against inflammatory disease caused by bacterial lipoproteins. Moreover, since TLRs are known to form homodimers and heterodimers with each other to recognize various bacterial components, it is likely that dimers formed with either TLR2 or TLR4 will be inhibited by unsaturated fatty acids, and stimulated by saturated fatty acids.

The complete disclosures of all references cited in this specification are hereby incorporated by reference. In the event of an otherwise irreconcilable conflict, however, the present specification shall control. Also incorporated by reference are the complete disclosures of the following items, none of which is prior art to the present invention: J. Y. Lee et al., “Saturated fatty acids, but not unsaturated fatty acids, induce the expression of cyclooxygenase-2 mediated through TOLL-like receptor 4,” Journal of Biological Chemistry, vol. 276, pp. 16683-16689 (2001); D. Hwang, “Modulation of the expression of cyclooxygenase-2 by fatty acids mediated through TOLL-like receptor 4 derived signalling pathways,” FASEB Journal, vol. 15, pp. 2556-2564 (2001); J. Y. Lee et al., “Differential modulation of Toll-like receptors by fatty acids - Preferential inhibition by n-3 polyunsaturated fatty acids,” manuscript in preparation; and K.-H. Shon, “The effects of dietary fatty acids and non-steroidal anti-inflammatory drugs on cyclooxygenase-2 expression,” a Dissertation submitted to Graduate faculty of Louisiana State University and Agricultural and Mechanical College for a degree in the Department of Food Science, August 2000.

I claim:

1. A method of ameliorating or preventing, in a mammal, the symptoms of a severe inflammatory disorder associated with activation of a Toll-like receptor, said method comprising administering to the mammal a therapeutically effective amount of one or more unsaturated fatty acids that are essentially free of saturated fatty acids.

2. A method as described in claim 1, wherein the disorder is selected from the group consisting of sepsis, septic shock, and endotoxemia.

3. A method as described in claim 1, wherein the Toll-like receptor is TLR4.

4. A method as described in claim 3, wherein the disorder is associated with activation of TLR4 caused by one or more compounds selected from the group consisting of lipopolysaccharide, Taxol, heat shock protein 60, and respiratory syncytial virus coat protein F.

5. A method as described in claim 1, wherein the Toll-like receptor is TLR2.

6. A method as described in claim 5, wherein the disorder is associated with activation of TLR2 caused by one or more compounds selected from the group consisting of bacterial lipoprotein, glycolipid, peptidoglycan, and zymosan.

7. A method as described in claim 1, wherein the unsaturated fatty acids are one or more fatty acids selected from the group consisting of eicosapentaenoic acid, docosahexaenoic acid, linoleic acid, conjugated linoleic acid, and oleic acid.

8. A method as described in claim 7, wherein the unsaturated fatty acid is docosahexaenoic acid.

9. A method as described in claim 1, wherein the unsaturated fatty acids comprise two or more unsaturated fatty acids selected from the group consisting of eicosapentaenoic acid, docosahexaenoic acid, linoleic acid, conjugated linoleic acid, and oleic acid.

10. A method as described in claim 1, additionally comprising administering to the mammal one or more compounds selected from the group consisting of radicicol, sesquiterpene lactones, rofecoxib; etoricoxib; NS-398; DuP-697; SC-58125; DFU; L-745,337; RS 57067; celecoxib; valdecoxib; meloxicam; flosulide; nimesulide; and parecoxib.

11. A method as described in claim 1, additionally comprising administering to the mammal a bactericidal amount of an antibiotic.

12. A method of ameliorating or preventing, in a mammal, the symptoms of a severe inflammatory disorder associated with activation of a Toll-like receptor, said method comprising administering to the mammal a therapeutically effective amount of a triglyceride comprising only unsaturated fatty acids or of a mixture of triglycerides comprising only unsaturated fatty acids in a solution essentially free of saturated fatty acids.

13. A method as described in claim 12, wherein the disorder is selected from the group consisting of sepsis, septic shock, and endotoxemia.

14. A method as described in claim 12, wherein the Toll-like receptor is TLR4.

15. A method as described in claim 14, wherein the disorder is associated with activation of TLR4 caused by one or more compounds selected from the group consisting of lipopolysaccharide, Taxol, heat shock protein 60, and respiratory syncytial virus coat protein F.

16. A method as described in claim 12, wherein the Toll-like receptor is TLR2.

17. A method as described in claim 16, wherein the disorder is associated with activation of TLR2 caused by one or more compounds selected from the group consisting of bacterial lipoprotein, glycolipid, peptidoglycan, and zymosan.

18. A method as described in claim 12, wherein the unsaturated fatty acids are one or more fatty acids selected from the group consisting of eicosapentaenoic acid, docosahexaenoic acid, linoleic acid, conjugated linoleic acid, and oleic acid.

19. A method as described in claim 18, wherein the unsaturated fatty acid is docosahexaenoic acid.

20. A method as described in claim 12, additionally comprising administering to the mammal one or more compounds selected from the group consisting of radicicol, sesquiterpene lactones, rofecoxib; etoricoxib; NS-398; DuP-697; SC-58125; DFU; L-745,337; RS 57067; celecoxib; valdecoxib; meloxicam; flosulide; nimesulide; and parecoxib.

21. A method as described in claim 12, additionally comprising administering to the mammal a bactericidal amount of an antibiotic.
